(19) United States
(12) Patent Application Publication
(10) Pub. No.: US 2025/0302621 A1
(43) Pub. Date: Oct. 2, 2025

(54) DELIVERY SYSTEM COMMISSURAL ALIGNMENT MARKERS

(71) Applicant: BOSTON SCIENTIFIC SCIMED, INC., Maple Grove, MN (US)

(72) Inventors: James Holmberg, Champlin, MN (US); Chenhao Fu, Plymouth, MN (US); James M. Anderson, Corcoran, MN (US); Kelsey Rae Cooper, Andover, MN (US); Levi Joel Wolterstorff, Saint Paul, MN (US); Dennis A Boismier, Shorewood, MN (US)

(73) Assignee: BOSTON SCIENTIFIC SCIMED, INC., Maple Grove, MN (US)

(21) Appl. No.: 19/097,398

(22) Filed: Apr. 1, 2025

Related U.S. Application Data
(60) Provisional application No. 63/573,319, filed on Apr. 2, 2024.

(51) Int. Cl. A61F 2/24; A61B 90/00

(52) U.S. Cl. CPC A61F 2/2436; A61B 90/39; A61B 2090/3966

(57) ABSTRACT

An implant delivery system includes an elongate shaft assembly including an implant holding portion configured to constrain the implant in a collapsed configuration. The shaft assembly includes a stent holder configured to engage the implant and a plurality of longitudinal marker elements. The marker elements are spaced distally from stent holder and configured to align commissure posts of the implant with native commissures under fluoroscopy. The shaft assembly may include a distal cap extending distal of the marker elements. The marker elements are disposed inward of the cap. Each marker element may include a non-longitudinal directional marker element. A method of delivering the implant includes configuring an imaging device to produce a 3-cusp view, advancing the delivery system adjacent the native valve, imaging to determine an initial orientation, rotating the delivery system to position a middle marker element in a posterior position; and deploying the implant.

### CROSS-REFERENCE TO RELATED APPLICATION

This application is a continuation of U.S. Patent Application Ser. No. 63/573,319, filed Apr. 2, 2024, entitled “DELIVERY SYSTEM COMMISSURAL ALIGNMENT MARKERS”, which is incorporated by reference herein in its entirety.

### TECHNICAL FIELD

The disclosure relates generally to medical devices and more particularly to medical devices that are adapted for implanting stents and medical devices including a stent component.

### BACKGROUND

A wide variety of intracorporeal medical devices have been developed for medical use including, artificial heart valves for repair or replacement of diseased heart valves. The artificial heart valves need to be precisely aligned relative to a native valve annulus when implanted. Of the known medical devices and methods, each has certain advantages and disadvantages. There is an ongoing need to provide alternative medical devices as well as alternative methods for manufacturing and using medical devices.

### SUMMARY

In one example, an implant delivery system for delivering a replacement heart valve implant to a native heart valve may comprise an elongate shaft assembly including an implant holding portion comprising a proximal sheath and a distal sheath. The implant holding portion may be configured to constrain a replacement heart valve implant in a radially collapsed configuration. The elongate shaft assembly may comprise a stent holder configured to engage an expandable framework of the replacement heart valve implant in the radially collapsed configuration, and a plurality of longitudinal marker elements configured to be visible under fluoroscopy with an imaging device, the plurality of longitudinal marker elements being spaced apart distally from the stent holder. The plurality of longitudinal marker elements may be configured to rotationally align commissure posts of the replacement heart valve implant with native valve commissures of the native heart valve under fluoroscopy with the imaging device when the replacement heart valve implant is shifted to a radially expanded configuration within the native heart valve.

In addition, or alternatively, to any example disclosed herein, each longitudinal marker element of the plurality of longitudinal marker elements comprises a non-longitudinal directional marker element extending therefrom.

In addition, or alternatively, to any example disclosed herein, each longitudinal marker element of the plurality of longitudinal marker elements is oriented parallel to a central longitudinal axis of the elongate shaft assembly and each non-longitudinal directional marker element extends laterally from its respective longitudinal marker element.

In addition, or alternatively, to any example disclosed herein, each longitudinal marker element of the plurality of longitudinal marker elements is oriented parallel to a central longitudinal axis of the elongate shaft assembly and each non-longitudinal directional marker element extends circumferentially from its respective longitudinal marker element.

In addition, or alternatively, to any example disclosed herein, each non-longitudinal directional marker element extends from a free end of its respective longitudinal marker element.

In addition, or alternatively, to any example disclosed herein, each non-longitudinal directional marker element extends from a medial portion of its respective longitudinal marker element.

In addition, or alternatively, to any example disclosed herein, each longitudinal element of the plurality of longitudinal marker elements is formed from a radiopaque material and the stent holder is formed from a different material than the plurality of longitudinal marker elements.

In addition, or alternatively, to any example disclosed herein, each longitudinal marker element of the plurality of longitudinal marker elements is fixedly attached to an annular ring that is disposed about a central longitudinal axis of the elongate shaft assembly.

In addition, or alternatively, to any example disclosed herein, the annular ring is spaced apart distally from the stent holder.

In addition, or alternatively, to any example disclosed herein, an implant delivery system for delivering a replacement heart valve implant to a native heart valve may comprise an elongate shaft assembly including an implant holding portion comprising a proximal sheath and a distal sheath. The implant holding portion may be configured to constrain a replacement heart valve implant in a radially collapsed configuration. The elongate shaft assembly may comprise a stent holder configured to engage a distal portion of an expandable framework of the replacement heart valve implant when the replacement heart valve implant is constrained within the implant holding portion in the radially collapsed configuration, a plurality of longitudinal marker elements disposed distal of the stent holder and configured to be visible under fluoroscopy, and a distal cap disposed at least partially distal of the stent holder and extending distal of the plurality of longitudinal marker elements. The plurality of longitudinal marker elements may be disposed radially inward of an outward facing surface of the distal cap. The plurality of longitudinal marker elements may be configured to rotationally align commissure posts of the replacement heart valve implant with native valve commissures of the native heart valve under fluoroscopy with the imaging device when the replacement heart valve implant is shifted to a radially expanded configuration within the native heart valve.

In addition, or alternatively, to any example disclosed herein, each longitudinal marker element of the plurality of longitudinal marker elements is fixedly attached to an annular ring disposed about a central longitudinal axis of the elongate shaft assembly.

In addition, or alternatively, to any example disclosed herein, each longitudinal marker element of the plurality of longitudinal marker elements extends distally from the annular ring.

In addition, or alternatively, to any example disclosed herein, each longitudinal marker element of the plurality of longitudinal marker elements extends proximally from the annular ring.

In addition, or alternatively, to any example disclosed herein, the distal cap is substantially transparent under fluoroscopy.

In addition, or alternatively, to any example disclosed herein, a method of delivering a replacement heart valve implant to a native heart valve may comprise configuring an imaging device to produce a 3-cusp view of the native heart valve under fluoroscopy in a first position and a cusp overlap view of the native heart valve under fluoroscopy in a second position; advancing an implant delivery system to a position adjacent the native heart valve, wherein the replacement heart valve implant is constrained within an implant holding portion of the implant delivery system, and the implant delivery system comprises a plurality of longitudinal marker elements, wherein each longitudinal marker element of the plurality of longitudinal marker elements comprises a non-longitudinal directional marker element extending therefrom; imaging the implant delivery system adjacent the native heart valve under fluoroscopy with the imaging device to determine an initial orientation of the replacement heart valve implant relative to the native heart valve in the 3-cusp view; thereafter, without switching from the 3-cusp view, rotating the implant delivery system in situ under fluoroscopy to position the plurality of longitudinal marker elements in a desired final orientation in the 3-cusp view, wherein in the desired final orientation under fluoroscopy in the 3-cusp view a middle longitudinal marker element of the plurality of longitudinal marker elements is disposed in a posterior position relative to other longitudinal marker elements of the plurality of longitudinal marker elements; and deploying the replacement heart valve implant within the native heart valve with the plurality of longitudinal marker elements in the desired final orientation.

In addition, or alternatively, to any example disclosed herein, the method may further comprise: prior to deploying the replacement heart valve implant, switching to the cusp overlap view to verify the plurality of longitudinal marker elements is in the desired final orientation.

In addition, or alternatively, to any example disclosed herein, under fluoroscopy in the desired final orientation, a single longitudinal marker element of the plurality of longitudinal marker elements is positioned farther away from a non-coronary cusp of the native heart valve than other longitudinal marker elements of the plurality of longitudinal marker elements in the cusp overlap view.

In addition, or alternatively, to any example disclosed herein, under fluoroscopy in the 3-cusp view, the non-longitudinal direction marker element associated with the middle longitudinal marker element of the plurality of longitudinal marker elements extends from the middle longitudinal marker element in a first direction when the middle longitudinal marker element is disposed in the posterior position relative to other longitudinal marker elements of the plurality of longitudinal marker elements, and the non-longitudinal direction marker element associated with the middle longitudinal marker element of the plurality of longitudinal marker elements extends from the middle longitudinal marker element extends in a second direction generally opposite the first direction when the middle longitudinal marker element is disposed in an anterior position relative to other longitudinal marker elements of the plurality of longitudinal marker elements.

In addition, or alternatively, to any example disclosed herein, each longitudinal marker element of the plurality of longitudinal marker elements is fixedly attached to an annular ring formed from a radiopaque material.

In addition, or alternatively, to any example disclosed herein, under fluoroscopy in the 3-cusp view, the annular ring is visualized as an oval if parallax is present and the annular ring is visualized as a straight line is parallax is not present.

The above summary of some embodiments, aspects, and/or examples is not intended to describe each disclosed embodiment or every implementation of the present disclosure. The figures and detailed description which follow more particularly exemplify these embodiments.

### BRIEF DESCRIPTION OF THE DRAWINGS

The disclosure may be more completely understood in consideration of the following detailed description in connection with the accompanying drawings, in which:

FIG. 1 is a partial cutaway view illustrating selected aspects of a replacement heart valve implant positioned within a native heart valve of a heart;

FIG. 2 is a partial cross-sectional view taken along the line 2-2 in FIG. 1 illustrating selected aspects of the heart;

FIG. 3 illustrates selected aspects of an implant delivery system for delivering a replacement heart valve implant;

FIG. 4 illustrates selected aspects of a portion of an implant delivery system according to the disclosure;

FIG. 5 illustrates selected aspects of a stent holder of the implant delivery system according to the disclosure;

FIG. 6 is an exploded view of selected aspects of an implant delivery system according to the disclosure;

FIG. 7A is a 3-cusp view of the native heart valve schematically illustrating relative positioning of selected aspects of the delivery system and the native heart valve during delivery of the replacement heart valve implant to the native heart valve;

FIG. 7B is a cusp overlap view of the native heart valve schematically illustrating relative positioning of selected aspects of the delivery system and the native heart valve during delivery of the replacement heart valve implant to the native heart valve;

FIG. 7C is a schematic illustration corresponding to the views of FIGS. 7A and 7B during delivery of the replacement heart valve implant to the native heart valve;

FIG. 8A is a 3-cusp view of the native heart valve schematically illustrating relative positioning of selected aspects of the delivery system and the native heart valve during delivery of the replacement heart valve implant to the native heart valve;

FIG. 8B is a cusp overlap view of the native heart valve schematically illustrating relative positioning of selected aspects of the delivery system and the native heart valve during delivery of the replacement heart valve implant to the native heart valve;

FIG. 8C is a is a schematic illustration corresponding to the views of FIGS. 8A and 8B during delivery of the replacement heart valve implant to the native heart valve;

FIG. 9 is an exploded view of selected aspects of an implant delivery system according to the disclosure;

FIG. 10A illustrates proximal-to-distal, tilted, and side views of a plurality of longitudinal marker elements of FIG. 9 oriented as in FIG. 7C;

FIG. 10B illustrates proximal-to-distal, tilted, and side views of the plurality of longitudinal marker elements of FIG. 9 oriented as in FIG. 8C;

FIG. 11 is an exploded view of selected aspects of an implant delivery system according to the disclosure;

FIG. 12A illustrates proximal-to-distal, tilted, and side views of a plurality of longitudinal marker elements of FIG. 11 oriented as in FIG. 7C;

FIG. 12B illustrates proximal-to-distal, tilted, and side views of the plurality of longitudinal marker elements of FIG. 11 oriented as in FIG. 8C;

FIG. 13 is an exploded view of selected aspects of an implant delivery system according to the disclosure;

FIG. 14A illustrates proximal-to-distal, tilted, and side views of a plurality of longitudinal marker elements of FIG. 13 oriented as in FIG. 7C;

FIG. 14B illustrates proximal-to-distal, tilted, and side views of the plurality of longitudinal marker elements of FIG. 13 oriented as in FIG. 8C;

FIG. 15 is an exploded view of selected aspects of an implant delivery system according to the disclosure;

FIG. 16A illustrates proximal-to-distal, tilted, and side views of a plurality of longitudinal marker elements of FIG. 15 oriented as in FIG. 7C;

FIG. 16B illustrates proximal-to-distal, tilted, and side views of the plurality of longitudinal marker elements of FIG. 15 oriented as in FIG. 8C;

FIG. 17 is an exploded view of selected aspects of an implant delivery system according to the disclosure;

FIG. 18A illustrates proximal-to-distal, tilted, and side views of a plurality of longitudinal marker elements of FIG. 17 oriented as in FIG. 7C; and

FIG. 18B illustrates proximal-to-distal, tilted, and side views of the plurality of longitudinal marker elements of FIG. 17 oriented as in FIG. 8C.

While aspects of the disclosure are amenable to various modifications and alternative forms, specifics thereof have been shown by way of example in the drawings and will be described in detail. It should be understood, however, that the intention is not to limit aspects of the disclosure to the particular embodiments described. On the contrary, the intention is to cover all modifications, equivalents, and alternatives falling within the spirit and scope of the disclosure.

### DETAILED DESCRIPTION

The following description should be read with reference to the drawings, which are not necessarily to scale, wherein like reference numerals indicate like elements throughout the several views. The detailed description and drawings are intended to illustrate but not limit the disclosure. Those skilled in the art will recognize that the various elements described and/or shown may be arranged in various combinations and configurations without departing from the scope of the disclosure. The detailed description and drawings illustrate example embodiments of the disclosure.

For the following defined terms, these definitions shall be applied, unless a different definition is given in the claims or elsewhere in this specification.

All numeric values are herein assumed to be modified by the term “about,” whether or not explicitly indicated. The term “about”, in the context of numeric values, generally refers to a range of numbers that one of skill in the art would consider equivalent to the recited value (e.g., having the same function or result). In many instances, the term “about” may include numbers that are rounded to the nearest significant figure. Other uses of the term “about” (e.g., in a context other than numeric values) may be assumed to have their ordinary and customary definition(s), as understood from and consistent with the context of the specification, unless otherwise specified.

The recitation of numerical ranges by endpoints includes all numbers within that range, including the endpoints (e.g., 1 to 5 includes 1, 1.5, 2, 2.75, 3, 3.80, 4, and 5).

Although some suitable dimensions, ranges, and/or values pertaining to various components, features and/or specifications are disclosed, one of skill in the art, incited by the present disclosure, would understand desired dimensions, ranges, and/or values may deviate from those expressly disclosed.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise. It is to be noted that to facilitate understanding, certain features of the disclosure may be described in the singular, even though those features may be plural or recurring within the disclosed embodiment(s). Each instance of the features may include and/or be encompassed by the singular disclosure(s), unless expressly stated to the contrary. For example, a reference to one feature may be equally referred to all instances and quantities beyond one of said feature unless clearly stated to the contrary. As such, it will be understood that the following discussion may apply equally to any and/or all components for which there are more than one within the device, etc. unless explicitly stated to the contrary.

Relative terms such as “proximal”, “distal”, “advance”, “retract”, variants thereof, and the like, may be generally considered with respect to the positioning, direction, and/or operation of various elements relative to a user/operator/manipulator of the device, wherein “proximal” and “retract” indicate or refer to closer to or toward the user and “distal” and “advance” indicate or refer to farther from or away from the user. In some instances, the terms “proximal” and “distal” may be arbitrarily assigned to facilitate understanding of the disclosure, and such instances will be readily apparent to the skilled artisan. Other relative terms, such as “upstream”, “downstream”, “inflow”, and “outflow” refer to a direction of fluid flow within a lumen, such as a body lumen, a blood vessel, or within a device. Still other relative terms, such as “axial”, “circumferential”, “longitudinal”, “lateral”, “radial”, etc. and/or variants thereof generally refer to direction and/or orientation relative to a central longitudinal axis of the disclosed structure or device.

The term “extent” may be understood to mean the greatest measurement of a stated or identified dimension, unless the extent or dimension in question is preceded by or identified as a “minimum”, which may be understood to mean the smallest measurement of the stated or identified dimension. For example, “outer extent” may be understood to mean an outer dimension, “radial extent” may be understood to mean a radial dimension, “longitudinal extent” may be understood to mean a longitudinal dimension, etc. Each instance of an “extent” may be different (e.g., axial, longitudinal, lateral, radial, circumferential, etc.) and will be apparent to the skilled person from the context of the individual usage. Generally, an “extent” may be considered a greatest possible dimension measured according to the intended usage, while a “minimum extent” may be considered a smallest possible dimension measured according to the intended usage. In some instances, an “extent” may generally be measured orthogonally within a plane and/or cross-section, but may be, as will be apparent from the particular context, measured differently—such as, but not limited to, angularly, radially, circumferentially (e.g., along an arc), etc.

The terms “monolithic” and “unitary” shall generally refer to an element or elements made from or consisting of a single structure or base unit/element. A monolithic and/or unitary element shall exclude structure and/or features made by assembling or otherwise joining multiple discrete structures or elements together.

It is noted that references in the specification to “an embodiment”, “some embodiments”, “other embodiments”, etc., indicate that the embodiment(s) described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it would be within the knowledge of one skilled in the art to implement the particular feature, structure, or characteristic in connection with other embodiments, whether or not explicitly described, unless clearly stated to the contrary. That is, the various individual elements described below, even if not explicitly shown in a particular combination, are nevertheless contemplated as being combinable or arrangeable with each other to form other additional embodiments or to complement and/or enrich the described embodiment(s), as would be understood by one of ordinary skill in the art.

For the purpose of clarity, certain identifying numerical nomenclature (e.g., first, second, third, fourth, etc.) may be used throughout the description and/or claims to name and/or differentiate between various described and/or claimed features. It is to be understood that the numerical nomenclature is not intended to be limiting and is exemplary only. In some embodiments, alterations of and deviations from previously used numerical nomenclature may be made in the interest of brevity and clarity. That is, a feature identified as a “first” element may later be referred to as a “second” element, a “third” element, etc. or may be omitted entirely, and/or a different feature may be referred to as the “first” element. The meaning and/or designation in each instance will be apparent to the skilled practitioner.

Additionally, it should be noted that in any given figure, some features may not be shown, or may be shown schematically, for clarity and/or simplicity. Additional details regarding some components and/or method steps may be illustrated in other figures in greater detail. The devices and/or methods disclosed herein may provide a number of desirable features and benefits as described in more detail below.

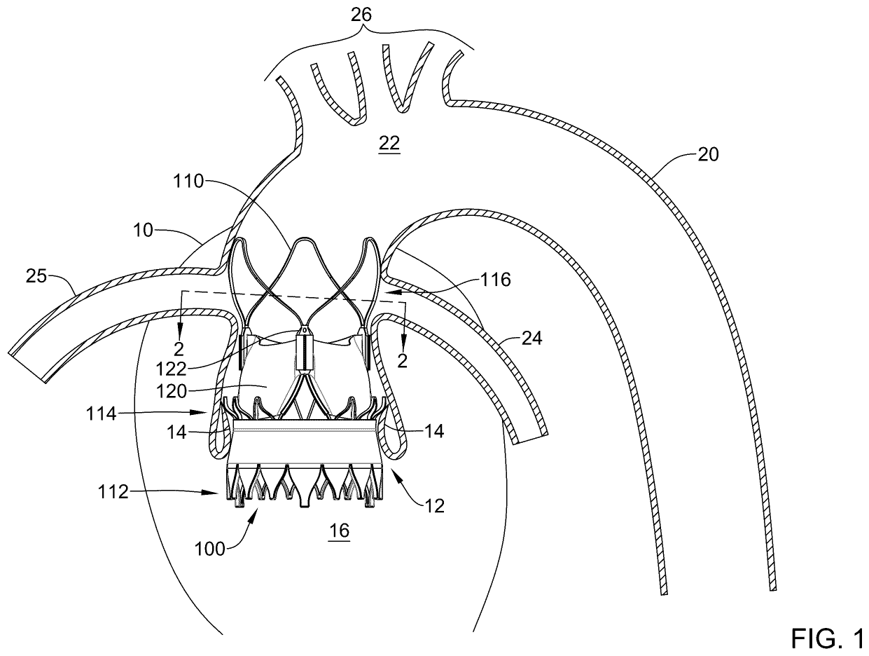

FIG. 1 illustrates a schematic partial cut-away view of a portion of a patient's heart 10 including the aortic valve 12 having native valve leaflets 14 disposed within and/or extending from the native valve annulus, a left ventricle 16, and certain connected vasculature, such as the aorta 20 connected to the aortic valve 12 of the patient's heart 10 by the aortic arch 22, the left coronary artery 24, the right coronary artery 25, and other large arteries 26 (e.g., subclavian arteries, carotid arteries, brachiocephalic artery) that extend from the aortic arch 22 to important internal organs. For the purpose of this disclosure, the discussion herein is directed toward use in treating a native heart valve such as the aortic valve 12 and will be so described in the interest of brevity. This, however, is not intended to be limiting as the skilled person will recognize that the following discussion may also apply to other heart valves, vessels, and/or treatment locations within a patient with no or minimal changes to the structure and/or scope of the disclosure.

FIG. 1 further illustrates selected aspects of a replacement heart valve implant 100 positioned within a native heart valve (e.g., the aortic valve 12). It should be appreciated that the replacement heart valve implant 100 can be any type of replacement heart valve (e.g., a mitral valve, an aortic valve, etc.). Some non-limiting examples of the replacement heart valve implant 100 may include the ACURATE NEO2™, the ACURATE PRIME™, and/or family members thereof from Boston Scientific. Other examples are also contemplated. In use, the replacement heart valve implant 100 may be implanted (e.g., surgically or through transcatheter delivery) in a mammalian heart. The replacement heart valve implant 100 can be configured to allow one-way flow through the replacement heart valve implant 100 from an inflow end to an outflow end.

The replacement heart valve implant 100 may include an expandable framework 110 defining a central lumen. In some embodiments, the expandable framework 110 may have a substantially circular cross-section. In some embodiments, the expandable framework 110 can have a non-circular (e.g., D-shaped, elliptical, etc.) cross-section. Some suitable but non-limiting examples of materials that may be used to form the expandable framework 110, including but not limited to metals and metal alloys, composites, ceramics, polymers, and the like, are described below. The replacement heart valve implant 100 and/or the expandable framework 110 may be configured to shift between a radially collapsed configuration and a radially expanded configuration. In some embodiments, the expandable framework 110 may be self-expanding. In some embodiments, the expandable framework 110 may be self-biased toward the radially expanded configuration. In some embodiments, the expandable framework 110 may be mechanically expandable. In some embodiments, the expandable framework 110 may be balloon expandable. Other configurations, including combinations thereof, are also contemplated.

In some embodiments, the expandable framework 110 may define a lower crown 112 proximate and/or at the inflow end, an upper crown 114 proximate and/or at the outflow end, and a plurality of stabilization arches 116 extending downstream from the outflow end. In some embodiments, the plurality of stabilization arches 116 may extend downstream of and/or away from the upper crown 114 in a direction opposite the lower crown 112. In some embodiments, the upper crown 114 may be disposed longitudinally and/or axially between the lower crown 112 and the plurality of stabilization arches 116. The expandable framework 110 may define a central lumen extending therethrough.

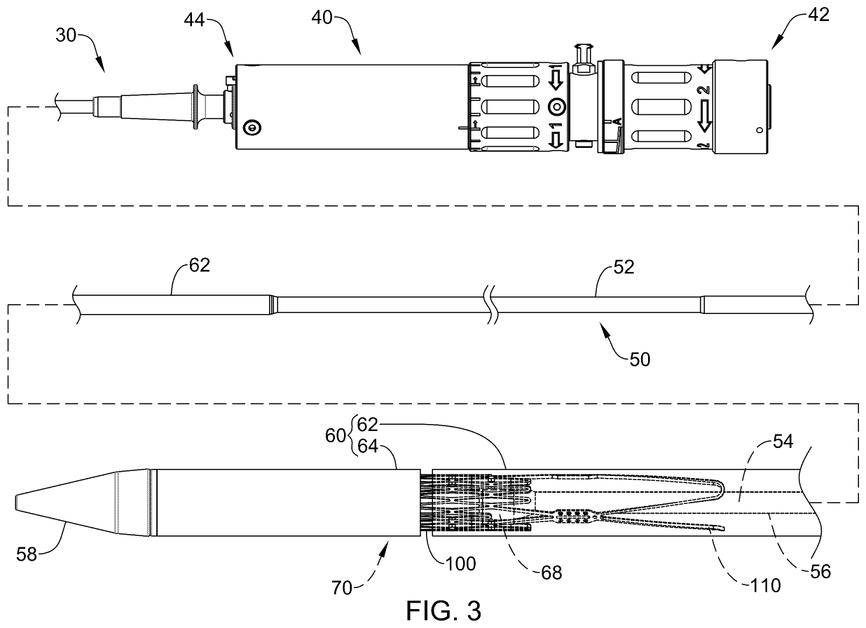

In some embodiments, the replacement heart valve implant 100 may include a proximal portion and a distal portion. In some embodiments, orientation of the replacement heart valve implant 100 may be related to an implant delivery system 30 (e.g., FIG. 3) and/or a direction of implantation relative to a target site (e.g., the native heart valve). In some embodiments, the proximal portion may include the outflow end and/or the plurality of stabilization arches 116. In some embodiments, the proximal portion may include the upper crown 114. In some embodiments, the distal portion may include the inflow end and/or the lower crown 112. Other configurations are also contemplated.

In some embodiments, the replacement heart valve implant 100 may include a plurality of valve leaflets 120 disposed within the central lumen. The plurality of valve leaflets 120 may be coupled, secured, and/or fixedly attached to the expandable framework 110 at a plurality of posts 122 to form and/or define a plurality of commissures. The plurality of valve leaflets 120 may be configured to shift between an open position and a closed position. The plurality of valve leaflets 120 may be configured to substantially restrict fluid flow through the replacement heart valve implant 100 in the closed position. The plurality of valve leaflets 120 may move apart from each other and/or radially outward within the central lumen in the open position to permit fluid flow through the replacement heart valve implant 100 and/or the central lumen.

In some embodiments, the plurality of valve leaflets 120 may be comprised of a polymer, such as a thermoplastic polymer. In some embodiments, the plurality of valve leaflets 120 may include at least 50 percent by weight of a polymer. In some embodiments, the plurality of valve leaflets 120 may be formed from porcine pericardium, bovine pericardium, or other tissue. Other configurations and/or materials are also contemplated.

In some embodiments, the replacement heart valve implant 100 may include an inner skirt disposed on and/or extending along an inner surface of the expandable framework 110. In at least some embodiments, the inner skirt may be fixedly attached to the expandable framework 110. The inner skirt may direct fluid, such as blood, flowing through the replacement heart valve implant 100 toward the plurality of valve leaflets 120. In at least some embodiments, the inner skirt may be fixedly attached to and/or integrally formed with the plurality of valve leaflets 120. The inner skirt may ensure the fluid flows through the central lumen of the replacement heart valve implant 100 and does not flow around the plurality of valve leaflets 120 when they are in the closed position.

In some embodiments, the replacement heart valve implant 100 may include an outer skirt disposed on and/or extending along an outer surface of the expandable framework 110. In some embodiments, the outer skirt may be disposed at and/or adjacent the lower crown. The outer skirt may ensure the fluid flows through the replacement heart valve implant 100 and does not flow around the replacement heart valve implant 100 (e.g., between the expandable framework 110 and the vessel wall).

In some embodiments, the inner skirt and/or the outer skirt may include a polymer, and/or may include at least 50 percent by weight of a polymer. In some embodiments, the inner skirt and/or the outer skirt may be substantially impervious to fluid. In some embodiments, the inner skirt and/or the outer skirt may be formed from a thin tissue (e.g., porcine pericardium, bovine pericardium, or other tissue, etc.), a coated fabric material, or a nonporous and/or impermeable fabric material. Other configurations are also contemplated. Some suitable but non-limiting examples of materials that may be used to form the inner skirt and/or the outer skirt including but not limited to polymers, composites, and the like, are described below.

In some embodiments, the inner skirt and/or the outer skirt may seal one of, some of, a plurality of, or each of a plurality of interstices formed in the expandable framework 110. In at least some embodiments, sealing the interstices may be considered to prevent fluid from flowing through the interstices of the expandable framework 110. In some embodiments, the inner skirt and/or the outer skirt may be attached to the expandable framework 110 using one or more methods including but not limited to tying with sutures or filaments, adhesive bonding, melt bonding, embedding or over molding, welding, etc.

In some embodiments, the expandable framework 110 and/or the replacement heart valve implant 100 may have an outer extent of about 23 millimeters (mm), about 25 mm, about 27 mm, about 30 mm, etc. in an unconstrained configuration (e.g., in the radially expanded configuration). In some embodiments, the expandable framework 110 and/or the replacement heart valve implant 100 may have an outer extent of about 10 mm, about 9 mm, about 8 mm, about 7 mm, about 6 mm, etc. in the radially collapsed configuration. Other configurations are also contemplated.

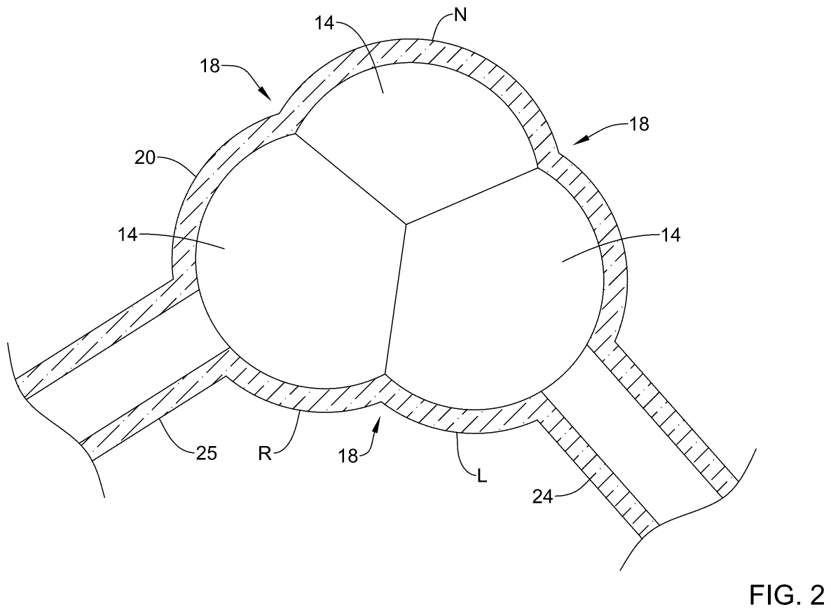

FIG. 2 is a partial cross-sectional view illustrating selected aspects of the native heart valve (e.g., the aortic valve 12) taken along the line 2-2 in FIG. 1. As shown in FIG. 2, the aorta 20 may include and/or may form three cusps proximate and/or immediately downstream of the native valve leaflets 14. The three cusps are the left coronary cusp L, the right coronary cusp R, and the non-coronary cusp N. The left coronary cusp L, the right coronary cusp R, and the non-coronary cusp N may form and/or resemble three lobes that come together at native valve commissures 18. The native valve leaflets 14 come together at the native valve commissures 18. The left coronary artery 24 opens into and/or extends from the left coronary cusp L. The right coronary artery 25 opens into and/or extends from the right coronary cusp R. No coronary artery is present in the non-coronary cusp N. For reference, the post (ref. 122) seen facing forward in FIG. 1 may be positioned adjacent a native valve commissure (ref. 18), such as that disposed between and/or formed by the left coronary cusp L and the right coronary cusp R of FIG. 2 (among other places).

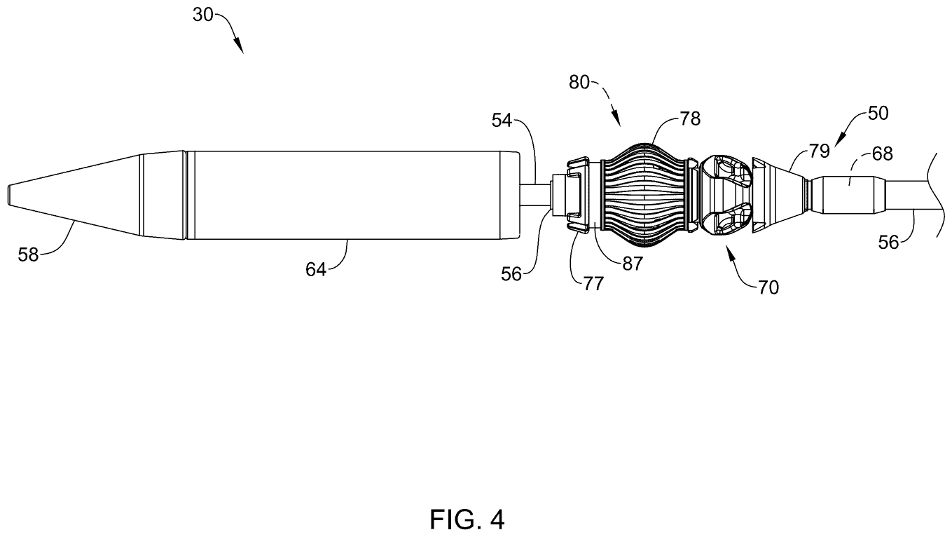

FIGS. 3-4 illustrate selected aspects of a replacement heart valve system including the replacement heart valve implant 100 and an implant delivery system 30 for delivering a replacement heart valve implant to a native heart valve (e.g., the aortic valve 12). The implant delivery system 30 may be compatible with and/or usable with the replacement heart valve implant 100. It should be noted that FIG. 3 includes at least one change of scale (e.g., all parts of the figure are not drawn to the same scale) to improve viewability and show additional detail of selected aspects of the implant delivery system 30. Additionally, the expandable framework 110 is shown in FIG. 3 in the radially collapsed configuration but some elements of the replacement heart valve implant 100 are not shown to improve clarity. In FIG. 4, the replacement heart valve implant 100 has been omitted.

The implant delivery system 30 may include a handle 40 and an elongate shaft assembly 50 extending distally from the handle 40. The handle 40 may include a first end 42 and a second end 44 opposite the first end 42. The elongate shaft assembly 50 may extend distally from the second end 44 of the handle 40. The handle 40 may include one or more rotatable knobs. In some embodiments, the one or more rotatable knobs may include a first rotatable knob and a second rotatable knob. In at least some embodiments, the first rotatable knob and/or the second rotatable knob may be configured to rotate about a central longitudinal axis of the implant delivery system 30 and/or the handle 40. Other configurations are also contemplated.

In some embodiments, a distal portion of the implant delivery system 30 and/or the elongate shaft assembly 50 may include an implant holding portion 60 configured to engage with and/or constrain the replacement heart valve implant 100 and/or the expandable framework 110 in the radially collapsed configuration. The elongate shaft assembly 50 may include an outer tubular member 52 extending distally from the handle 40 and an inner shaft 54 (e.g., FIG. 4) extending distally from the handle 40 within the outer tubular member 52 to a distal tip 58 disposed distal of the implant holding portion 60. In some embodiments, the implant holding portion 60 may comprise a proximal sheath 62 and a distal sheath 64. In some embodiments, the proximal sheath 62 and/or the distal sheath 64 may be formed from a polymeric material. In some embodiments, the proximal sheath 62 and/or the distal sheath 64 may include a reinforcing structure disposed therein and/or thereon. In some embodiments, the reinforcing structure may be a coil, a mesh, one or more filaments, bands, or strips, or another suitable structure. Other configurations are also contemplated.

In some embodiments, the inner shaft 54 may be slidably disposed within a lumen of the outer tubular member 52. In some embodiments, the elongate shaft assembly 50 may include an intermediate tubular member 56 disposed within and/or radially inward of the outer tubular member 52 and about and/or radially outward of the inner shaft 54. In at least some embodiments, the inner shaft 54 and the outer tubular member 52 are each axially translatable relative to the intermediate tubular member 56 independently of each other. For example, the inner shaft 54 may be translated relative to the intermediate tubular member 56 without translating the outer tubular member 52 relative to the intermediate tubular member 56, and vice versa.

In some embodiments, the proximal sheath 62 may be fixedly attached to the outer tubular member 52. In some embodiments, the proximal sheath 62 may be fixedly attached to and/or may extend distally from a distal end of the outer tubular member 52. In some embodiments, the distal sheath 64 and/or the distal tip 58 may be fixedly attached to the inner shaft 54. In some embodiments, the distal sheath 64 may be fixedly attached to the distal tip 58. In some embodiments, the distal sheath 64 may extend proximally from the distal tip 58. In some embodiments, the inner shaft 54 may include and/or at least partially define a guidewire lumen extending therethrough. In some embodiments, the guidewire lumen may extend through the handle 40.

In some embodiments, the handle 40 may be configured to manipulate and/or translate the proximal sheath 62 and/or the distal sheath 64 relative to each other using the first rotatable knob and/or the second rotatable knob. In some embodiments, the handle 40 may be configured to manipulate and/or translate the inner shaft 54 and/or the distal sheath 64 relative to the elongate shaft assembly 50, the outer tubular member 52, the intermediate tubular member 56, and/or the proximal sheath 62. In some embodiments, the handle 40 may be configured to manipulate and/or translate the outer tubular member 52 and/or the proximal sheath 62 relative to the elongate shaft assembly 50, the inner shaft 54, the intermediate tubular member 56, and/or the distal sheath 64.

During delivery of the replacement heart valve implant 100 to a treatment site (e.g., the native heart valve, the aortic valve 12, etc.), the replacement heart valve implant 100 may be disposed at least partially within the proximal sheath 62 and/or the distal sheath 64 in the radially collapsed configuration in a closed configuration of the implant holding portion 60. In some embodiments, the proximal sheath 62 and/or the distal sheath 64 may collectively define the implant holding portion 60 of the implant delivery system 30. In some embodiments, the implant holding portion 60 may be configured to constrain the replacement heart valve implant 100 in the radially collapsed configuration when the implant holding portion 60 is in the closed configuration. In some embodiments, the replacement heart valve implant 100 may be releasably coupled to and/or releasably engaged with the intermediate tubular member 56 and/or a stent holder 70 when the replacement heart valve implant 100 is constrained within the implant holding portion 60 of the implant delivery system 30 in the radially collapsed configuration.

In some embodiments, the proximal sheath 62 may be configured to cover the proximal portion of the replacement heart valve implant 100 in the radially collapsed configuration when the implant holding portion 60 is in the closed configuration, and the distal sheath 64 may be configured to cover the distal portion of the replacement heart valve implant 100 in the radially collapsed configuration when the implant holding portion 60 is in the closed configuration. In some embodiments, the replacement heart valve implant 100 and/or the expandable framework 110 may be constrained in the radially collapsed configuration by the proximal sheath 62 and the distal sheath 64 in the closed configuration of the implant holding portion 60. In some embodiments, the proximal sheath 62 may be disposed adjacent to the distal sheath 64 in the closed configuration. In some embodiments, the proximal sheath 62 may abut the distal sheath 64 in the closed configuration. In some embodiments, the proximal sheath 62 may be axially spaced apart from the distal sheath 64 in the closed configuration. In some embodiments, the proximal sheath 62 may be axially spaced apart from the distal sheath 64 in the closed configuration by less than 20% of an overall length of the replacement heart valve implant 100 and/or the expandable framework 110. In some embodiments, the proximal sheath 62 may be axially spaced apart from the distal sheath 64 in the closed configuration by less than 15% of an overall length of the replacement heart valve implant 100 and/or the expandable framework 110. In some embodiments, the proximal sheath 62 may be axially spaced apart from the distal sheath 64 in the closed configuration by less than 10% of an overall length of the replacement heart valve implant 100 and/or the expandable framework 110. In some embodiments, the proximal sheath 62 may be axially spaced apart from the distal sheath 64 in the closed configuration by less than 5% of an overall length of the replacement heart valve implant 100 and/or the expandable framework 110. Other configurations are also contemplated.

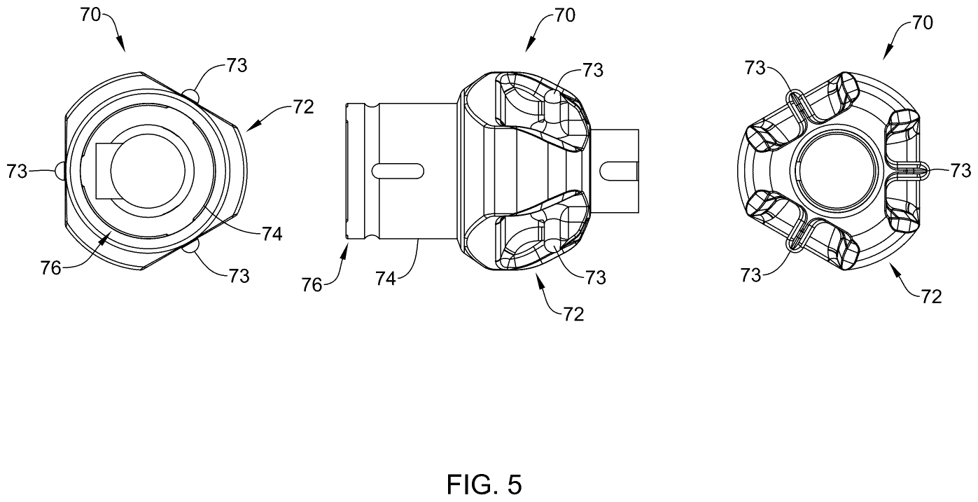

In some embodiments, the implant holding portion 60 and/or the elongate shaft assembly 50 may include the stent holder 70, which is shown in more detail in FIG. 5. In at least some embodiments, the stent holder 70 may be fixedly attached to the elongate shaft assembly 50. In some embodiments, the stent holder 70 may be fixedly attached to the intermediate tubular member 56 of the elongate shaft assembly 50. In some embodiments, the stent holder 70 may be integrally formed with the elongate shaft assembly 50 and/or the intermediate tubular member 56. In some embodiments, the stent holder 70 may be configured to engage the expandable framework 110 in the radially collapsed configuration and/or when the replacement heart valve implant 100 is constrained within the implant holding portion 60 of the implant delivery system 30. In some embodiments, the stent holder 70 may include at least one projection 73 (e.g., FIG. 5) configured to engage the expandable framework 110 in the radially collapsed configuration.

The implant delivery system 30 and/or the elongate shaft assembly 50 may include a primary visual indicator 68 configured and/or adapted to be visible under fluoroscopy with an imaging device. Other imaging means suitable for use with transcatheter surgical procedures are also contemplated. The implant delivery system 30 and/or the primary visual indicator 68 may be configured to cooperate with the imaging device to position the replacement heart valve implant 100 at a desired insertion depth within the native heart valve (e.g., the aortic valve 12).

In use, the implant delivery system 30 may be advanced percutaneously through the vasculature to a position adjacent to the treatment site (e.g., the native valve annulus). For example, the implant delivery system 30 may be advanced through the vasculature and across the aortic arch 22 to a position adjacent to the native heart valve (e.g., the aortic valve 12). Alternative approaches to treat a defective aortic valve and/or other heart valve(s) are also contemplated with the implant delivery system 30.

The desired insertion depth may be selected to maximize radially outward force of the expandable framework 110 within the native heart valve (e.g., the aortic valve 12). Positioning the replacement heart valve implant 100 at the desired insertion depth and/or within a maximum tolerance from the desired insertion depth, the replacement heart valve implant 100 and/or the expandable framework 110 may exhibit optimal arching within the native heart valve (e.g., the aortic valve 12) and thereby prevent migration of the replacement heart valve implant 100 and/or the expandable framework 110 downstream (or upstream).

Positioning the replacement heart valve implant 100 and/or the expandable framework 110 within the native heart valve (e.g., the aortic valve 12) may be accomplished by locating the primary visual indicator 68 relative to the native heart valve (e.g., the aortic valve 12). During visualization, the native heart valve (e.g., the aortic valve 12) may be identified and/or visualized under fluoroscopy using known means and/or methods, such as contrast injection.

FIG. 4 illustrates selected aspects of the implant delivery system 30 of FIG. 3 in accordance with the disclosure. For improved clarity, the replacement heart valve implant 100 and some portions of the implant delivery system 30 are not shown. For example, in FIG. 4, the distal sheath 64 is shown while the proximal sheath 62 is not shown.

In some embodiments, the primary visual indicator 68 may be fixedly attached to the elongate shaft assembly 50 and/or the intermediate tubular member 56. In some embodiments, the primary visual indicator 68 may be a marker band embedded within the elongate shaft assembly 50. In some embodiments, the primary visual indicator 68 may be fixedly attached to the elongate shaft assembly 50 and/or the intermediate tubular member 56 by a shrink wrap or by an adhesive element. Other configurations are also contemplated. As discussed herein, the primary visual indicator 68 may be configured and/or adapted to be visible under fluoroscopy with the imaging device. The primary visual indicator 68 may be formed from a radiopaque material. Some suitable but non-limiting examples of radiopaque materials for the primary visual indicator 68 are described below.

In some embodiments, the implant delivery system 30 and/or the elongate shaft assembly 50 may include the stent holder 70 configured to engage the expandable framework 110 of the replacement heart valve implant 100 in the radially collapsed configuration and/or when the replacement heart valve implant 100 is constrained within the implant holding portion 60 of the implant delivery system 30. The stent holder 70 is shown in more detail in FIG. 5. As shown in FIG. 5, the stent holder 70 may include a body 74, a first end portion 72 extending proximally from the body 74, and a second end portion 76 disposed opposite the first end portion 72. In some embodiments, the first end portion 72 may have a generally bulbous shape. In some embodiments, the stent holder 70 may be configured and/or adapted to be visible under fluoroscopy. In some embodiments, the stent holder 70 may be formed from stainless steel. Some suitable but non-limiting materials for the stent holder 70 and/or components or elements thereof are described below.

In some embodiments, an outermost radial extent of the first end portion 72 of the stent holder 70 may be disposed proximate a distal end of the first end portion 72 of the stent holder 70. In some embodiments, the first end portion 72 of the stent holder 70 may be tapered radially inward in a proximal direction from the outermost radial extent of the stent holder 70. In some embodiments, the stent holder 70 may include a lumen extending longitudinally and/or axially therethrough. In at least some embodiments, at least a portion of the elongate shaft assembly 50 may extend longitudinally and/or axially through the lumen of the stent holder 70.

The first end portion 72 may be configured and/or adapted to engage the expandable framework 110 of the replacement heart valve implant 100 (not shown) in the radially collapsed configuration and/or when the replacement heart valve implant 100 is constrained within the implant holding portion 60 of the implant delivery system 30. In some embodiments, the first end portion 72 may include at least one projection 73 configured and/or adapted to engage the expandable framework 110 of the replacement heart valve implant 100 in the radially collapsed configuration and/or when the replacement heart valve implant 100 is constrained within the implant holding portion 60 of the implant delivery system 30.

In some embodiments, at least one projection 73 may be configured and/or adapted to engage a distal portion and/or the lower crown 112 of the expandable framework 110 of the replacement heart valve implant 100 (not shown) in the radially collapsed configuration and/or when the replacement heart valve implant 100 is constrained within the implant holding portion 60 of the implant delivery system 30. In some embodiments, the at least one projection 73 may extend radially outward from the first end portion 72 of the stent holder 70. In some embodiments, the at least one projection 73 may extend radially outward through the expandable framework 110 of the replacement heart valve implant 100 in the radially collapsed configuration and/or when the replacement heart valve implant 100 is constrained within the implant holding portion 60 of the implant delivery system 30.

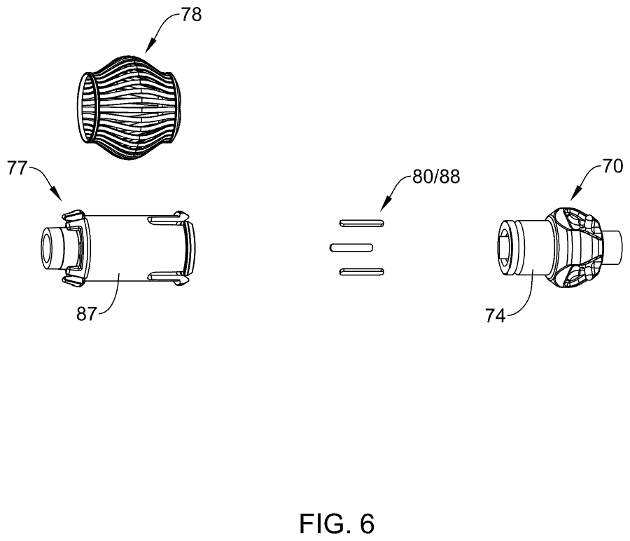

The elongate shaft assembly 50 may comprise a plurality of longitudinal marker elements 80 (e.g., FIGS. 6 and 9-18B) configured to be visible under fluoroscopy with an imaging device. In some embodiments, the plurality of longitudinal marker elements 80 may include two longitudinal marker elements, three longitudinal marker elements, four longitudinal marker elements, etc. In one example, the plurality of longitudinal marker elements 80 may include exactly three longitudinal marker elements. Other configurations are also contemplated.

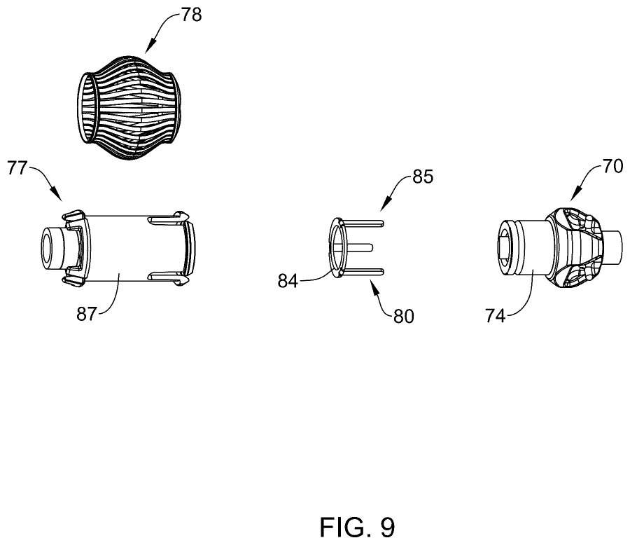

In some embodiments, each longitudinal marker element of the plurality of longitudinal marker elements 80 may be fixedly attached to an annular ring 84 (e.g., FIGS. 9-18B) that is disposed about the central longitudinal axis of the elongate shaft assembly 50. In some embodiments, each longitudinal marker element of the plurality of longitudinal marker elements 80 may be integrally formed with and/or may be monolithic with the annular ring 84. In some embodiments, the plurality of longitudinal marker elements 80 and the annular ring 84 may collectively define and/or be referred to as a commissure marker 85 (e.g., FIGS. 9-18B). Some example configurations for the plurality of longitudinal marker elements 80, the annular ring 84, and/or the commissure marker 85 are shown in FIGS. 9-18B and are described in more detail below.

In some embodiments, the plurality of longitudinal marker elements 80 may be disposed distally of the body 74 of the stent holder 70. In some embodiments, the annular ring 84 may be coupled and/or attached to the stent holder 70 and/or the body 74 of the stent holder 70. In some embodiments, the annular ring 84 may be disposed adjacent the distal end of the stent holder 70 and/or the body 74 of the stent holder 70. In some embodiments, the annular ring 84 may be sandwiched between the stent holder 70 and another component of the elongate shaft assembly 50 (e.g., the distal cap 77 described herein, etc.). In at least some embodiments, the plurality of longitudinal marker elements 80 may be spaced apart distally from a distal end of the stent holder 70 and/or the body 74 of the stent holder 70. In some embodiments, the annular ring 84 may be spaced apart distally from the distal end of the stent holder 70 and/or the body 74 of the stent holder 70. In some embodiments, the plurality of longitudinal marker elements 80 may extend distally from the stent holder 70. In some embodiments, the plurality of longitudinal marker elements 80 may extend distally from the annular ring 84. In some embodiments, the plurality of longitudinal marker elements 80 may extend proximally from the annular ring 84. In some embodiments, the plurality of longitudinal marker elements 80 may extend proximally and distally from the annular ring 84. Other configurations are also contemplated.

In some embodiments, the plurality of longitudinal marker elements 80 may be oriented parallel to a central longitudinal axis of the elongate shaft assembly 50. In some embodiments, the plurality of longitudinal marker elements 80 may be spaced radially outward from the inner shaft 54 and/or the intermediate tubular member 56 of the elongate shaft assembly 50. In some embodiments, the plurality of longitudinal marker elements 80 may be disposed radially closer to the distal sheath 64 than the central longitudinal axis of the elongate shaft assembly 50. In some embodiments, the plurality of longitudinal marker elements 80 may be disposed a first radial distance from the central longitudinal axis of the elongate shaft assembly 50 and a second radial distance from the distal sheath 64 and/or an inner surface of the distal sheath 64, wherein the second radial distance is less than the first radial distance. In some embodiments, the plurality of longitudinal marker elements 80 may be equally spaced apart from each other circumferentially about the central longitudinal axis of the elongate shaft assembly 50.

In some embodiments, each longitudinal marker element of the plurality of longitudinal marker elements 80 may be oriented parallel to a central longitudinal axis of the elongate shaft assembly 50. In some embodiments, each longitudinal marker element of the plurality of longitudinal marker elements 80 may be spaced radially outward from the inner shaft 54 and/or the intermediate tubular member 56 of the elongate shaft assembly 50. In some embodiments, each longitudinal marker element of the plurality of longitudinal marker elements 80 may be disposed radially closer to the distal sheath 64 than the central longitudinal axis of the elongate shaft assembly 50. In some embodiments, each longitudinal marker element of the plurality of longitudinal marker elements 80 may be disposed a first radial distance from the central longitudinal axis of the elongate shaft assembly 50 and a second radial distance from the distal sheath 64 and/or an inner surface of the distal sheath 64, wherein the second radial distance is less than the first radial distance. In some embodiments, each longitudinal marker element of the plurality of longitudinal marker elements 80 may be equally spaced apart from each other circumferentially about the central longitudinal axis of the elongate shaft assembly 50.

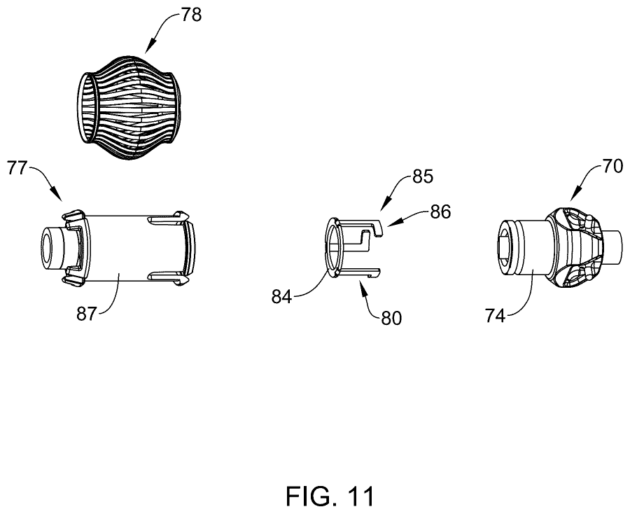

In some embodiments, each longitudinal marker element of the plurality of longitudinal marker elements 80 may comprise a non-longitudinal directional marker element 86 (e.g., FIGS. 11-14B, 17-18B) extending therefrom. In some embodiments, each non-longitudinal directional marker element 86 of the plurality of longitudinal marker elements 80 may be fixedly attached to its respective longitudinal marker element. In at least some embodiments, each non-longitudinal directional marker element 86 of the plurality of longitudinal marker elements 80 may be integrally formed with and/or may be monolithically formed with respective longitudinal marker element.

In some embodiments, each non-longitudinal directional marker element 86 of the plurality of longitudinal marker elements 80 may extend laterally from its respective longitudinal marker element. In some embodiments, each non-longitudinal directional marker element 86 of the plurality of longitudinal marker elements 80 may extend circumferentially from its respective longitudinal marker element about the central longitudinal axis of the elongate shaft assembly 50. In some embodiments, each non-longitudinal directional marker element 86 of the plurality of longitudinal marker elements 80 may be curved and/or may extend in an arc from its respective longitudinal marker element about the central longitudinal axis of the elongate shaft assembly 50. In some embodiments, each non-longitudinal directional marker element 86 of the plurality of longitudinal marker elements 80 may extend from a free end of its respective longitudinal marker element. In some alternative embodiments, each non-longitudinal directional marker element 86 of the plurality of longitudinal marker elements 80 may extend from a medial portion of its respective longitudinal marker element. Other configurations are also contemplated.

In some embodiments, the plurality of longitudinal marker elements 80 may be disposed within the elongate shaft assembly 50. In some alternative embodiments, the plurality of longitudinal marker elements 80 may be embedded within and/or fixedly attached to the elongate shaft assembly 50. In some further alternative embodiments, the plurality of longitudinal marker elements 80 may extend from and/or may be disposed proximal of the first end portion 72 of the stent holder 70. Other configurations are also contemplated.

In some embodiments, the plurality of longitudinal marker elements 80 may be longitudinally and/or axially aligned with the at least one projection 73 of the stent holder 70. In some embodiments, the plurality of longitudinal marker elements 80 may be configured and/or adapted to align with the plurality of posts 122 and/or the plurality of commissures of the replacement heart valve implant 100. In some embodiments, the plurality of longitudinal marker elements 80 may be configured and/or adapted to be visible under fluoroscopy.

In some embodiments, the plurality of longitudinal marker elements 80, the annular ring 84, and/or the commissure marker 85 may be formed from a radiopaque material. In some embodiments, the plurality of longitudinal marker elements 80, the annular ring 84, and/or the commissure marker 85 may be formed from a highly radiopaque material. In one non-limiting example, the plurality of longitudinal marker elements 80, the annular ring 84, and/or the commissure marker 85 may be formed from tungsten. In other non-limiting examples, the plurality of longitudinal marker elements 80, the annular ring 84, and/or the commissure marker 85 may be formed from tantalum, platinum, iridium, gold, silver, iodine, or combinations and/or alloys thereof. Other materials are also contemplated. In some embodiments, the stent holder 70 may be formed from a different material than the plurality of longitudinal marker elements 80, the annular ring 84, and/or the commissure marker 85. In at least some embodiments, the plurality of longitudinal marker elements 80, the annular ring 84, and/or the commissure marker 85 may be more radiopaque, and/or may appear darker or more defined, under fluoroscopy than the stent holder 70. Accordingly, the plurality of longitudinal marker elements 80, the annular ring 84, and/or the commissure marker 85 may “stand out” and/or may be more visible to the practitioner under fluoroscopy during the procedure than the replacement heart valve implant 100 (e.g., FIG. 1), such as the expandable framework 110 (e.g., FIG. 1), and/or other components and/or elements of the implant delivery system 30, such as the elongate shaft assembly 50, the stent holder 70, the proximal sheath 62, the distal sheath 64, etc.

In some embodiments, the plurality of longitudinal marker elements 80, the annular ring 84, and/or the commissure marker 85 may be configured and/or adapted to align the implant delivery system 30, the stent holder 70, and/or the plurality of posts 122 (e.g., FIG. 1) and the plurality of the commissures of the replacement heart valve implant 100 (e.g., FIG. 1) with the native valve commissures 18 of the native heart valve (e.g., the aortic valve 12) under fluoroscopy. In some embodiments, the plurality of longitudinal marker elements 80, the annular ring 84, and/or the commissure marker 85 may be configured and/or adapted to rotationally align the implant delivery system 30, the stent holder 70, and/or the plurality of posts 122 (e.g., FIG. 1) and the plurality of commissures of the replacement heart valve implant 100 (e.g., FIG. 1) with the native valve commissures 18 of the native heart valve (e.g., the aortic valve 12) under fluoroscopy.

In some embodiments, the distal sheath 64 may extend proximally from the distal tip 58, as seen in FIG. 4. The implant delivery system 30 and/or the elongate shaft assembly 50 may include a distal cap 77 disposed distal of at least a portion of the stent holder 70. In some embodiments, the distal cap 77 may be fixedly attached adjacent to, at, and/or to a distal end of the intermediate tubular member 56. In some embodiments, the distal cap 77 may be disposed at least partially distal of the stent holder 70 and/or the body 74 (e.g., FIG. 5) of the stent holder 70. In some embodiments, the distal cap 77 may extend distal of the plurality of longitudinal marker elements 80, the annular ring 84, and/or the commissure marker 85 (not shown in FIG. 4). In some embodiments, a proximal end of the distal cap 77 may extend proximal of the plurality of longitudinal marker elements 80, the annular ring 84, and/or the commissure marker 85.

In some embodiments, at least a portion of the distal cap 77 may be disposed radially outward of and/or axially overlapping the plurality of longitudinal marker elements 80, the annular ring 84, and/or the commissure marker 85. In some embodiments, the plurality of longitudinal marker elements 80 may be disposed radially inward of a radially outward facing surface 87 of the distal cap 77. In at least some embodiments, the plurality of longitudinal marker elements 80, the annular ring 84, and/or the commissure marker 85 may be disposed radially outward of the intermediate tubular member 56. In some embodiments, the plurality of longitudinal marker elements 80, the annular ring 84, and/or the commissure marker 85 may be disposed within the distal cap 77 and radially outward of the intermediate tubular member 56. In some embodiments, at least a portion of the annular ring 84 may be disposed radially inward of the outward facing surface of the distal cap 77. In some embodiments, the commissure marker 85, in its entirety, may be disposed radially inward of the outward facing surface of the distal cap 77.

In some embodiments, the annular ring 84 may be disposed proximate and/or adjacent a distal end of the distal cap 77. In at least some embodiments where the annular ring 84 is disposed proximate and/or adjacent the distal end of the distal cap 77, the plurality of longitudinal marker elements 80 may extend proximally from the annular ring 84. In some embodiments, the annular ring 84 may be disposed proximate and/or adjacent a proximal end of the distal cap 77. In at least some embodiments where the annular ring 84 is disposed proximate and/or adjacent the proximal end of the distal cap 77, the plurality of longitudinal marker elements 80 may extend distally from the annular ring 84. In some embodiments, the annular ring 84 may be disposed along a medial portion of the distal cap 77. In some embodiments, the annular ring 84 may be disposed between the stent holder 70 and the distal cap 77 with the plurality of longitudinal marker elements 80 extending distally from the annular ring 84. In some embodiments, the annular ring 84 may be disposed between the first end portion 72 of the stent holder 70 and the distal cap 77. In some embodiments, the annular ring 84 may be disposed between the second end portion 76 of the stent holder 70 and the distal cap 77 with the plurality of longitudinal marker elements 80 extending distally from the annular ring 84. In some embodiments where the annular ring 84 is disposed along the medial portion of the distal cap 77, the plurality of longitudinal marker elements 80 may extend proximally and/or distally from the annular ring 84. Other configurations are also contemplated.

In some embodiments, the implant delivery system 30 and/or the elongate shaft assembly 50 may include a cage 78 disposed radially outward of and/or extending radially outward from the elongate shaft assembly 50, the distal cap 77, the radially outward facing surface 87 of the distal cap 77, and/or the stent holder 70, as seen in FIG. 4. In some embodiments, the cage 78 may be configured to substantially center the distal sheath 64 over and/or around the elongate shaft assembly 50 and/or the replacement heart valve implant 100 (not shown) as the distal sheath 64 is moved from an open configuration to the closed configuration. Other configurations are also contemplated.

The cage 78 may be positioned radially outward of and axially overlapping the plurality of longitudinal marker elements 80. In some embodiments, the cage 78 may be positioned radially outward of and axially overlapping the body 74 of the stent holder 70. In some embodiments, the cage 78 may be positioned radially outward of and axially overlapping the radially outward facing surface 87 of the distal cap 77. In some embodiments, the distal cap 77 may include a flanged portion extending radially outward from the central longitudinal axis of the elongate shaft assembly 50. In some embodiments, the flanged portion of the distal cap 77 and the first end portion 72 of the stent holder 70 may cooperate to axially retain the cage 78 in place over the second end portion 76 of the stent holder 70, the outward facing surface of the distal cap 77, and/or the plurality of longitudinal marker elements 80. In some embodiments, the distal cap 77 may include a proximal flanged portion extending radially outward from the central longitudinal axis of the elongate shaft assembly 50 and a distal flanged portion extending radially outward from the central longitudinal axis of the elongate shaft assembly 50 extending radially outward from the central longitudinal axis of the elongate shaft assembly 50. In some embodiments, the proximal flanged portion of the distal cap 77 and the distal flanged portion of the distal cap 77 may cooperate to axially retain the cage 78 in place over the second end portion 76 of the stent holder 70, the outward facing surface of the distal cap 77, and/or the plurality of longitudinal marker elements 80.

In at least some embodiments, the distal cap 77 and the cage 78 may be formed from a fluoroscopically transparent (or fluoroscopically translucent) material such that the plurality of longitudinal marker elements 80, the annular ring 84, and/or the commissure marker 85 is clearly visible under fluoroscopy. Accordingly, the distal cap 77 and the cage 78 may be substantially transparent under fluoroscopy. In some embodiments, the distal cap 77 and the cage 78 may be formed from a polymeric material. In some embodiments, the elongate shaft assembly 50 may include at least one fluoroscopically transparent (or fluoroscopically translucent) component disposed radially outward of the plurality of longitudinal marker elements 80, the annular ring 84, and/or the commissure marker 85 and radially inward of the distal sheath 64 of the implant holding portion 60. In some embodiments, the plurality of longitudinal marker elements 80, the annular ring 84, and/or the commissure marker 85 may not be visible to the naked eye, even if the replacement heart valve implant 100 is not present and the implant holding portion 60 is in the open configuration (e.g., the distal sheath 64 has been translated distally relative to the intermediate tubular member 56 and/or the stent holder 70). In some embodiments, the plurality of longitudinal marker elements 80, the annular ring 84, and/or the commissure marker 85 is only visible under fluoroscopy.

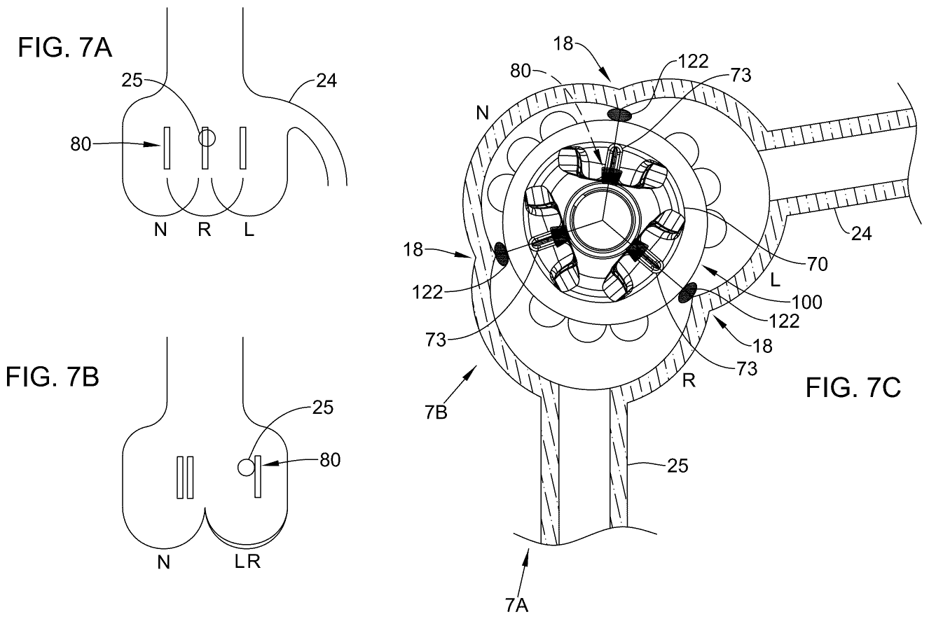

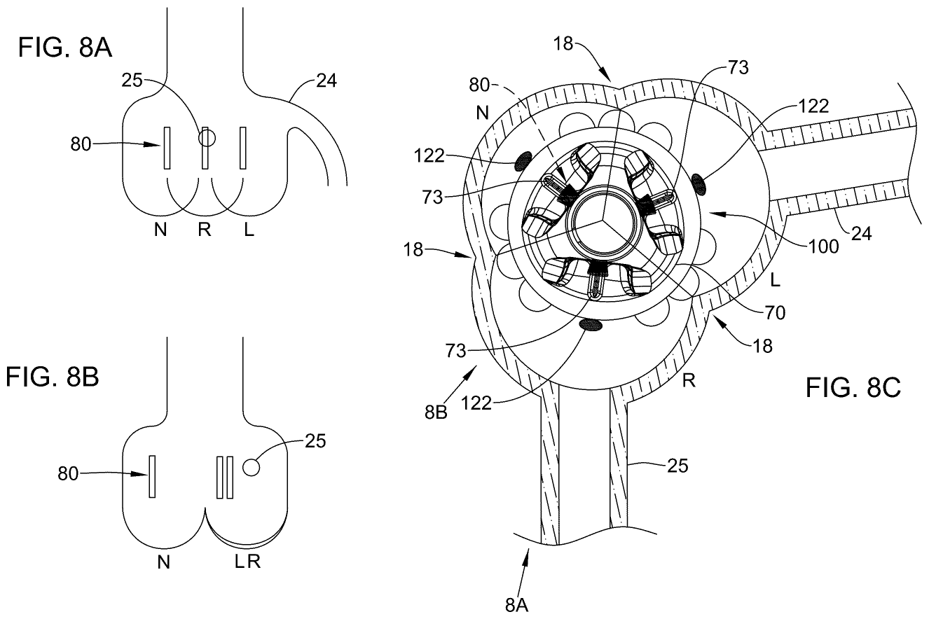

The plurality of longitudinal marker elements 80 may be clearly visible inside the cage 78 and the distal sheath 64 under fluoroscopy. Accordingly, the plurality of longitudinal marker elements 80 may be used to orient the replacement heart valve implant 100 (e.g., FIGS. 1, 7C, 8C) relative to the native heart valve (e.g., the aortic valve 12) under fluoroscopy such that the plurality of posts 122 (e.g., FIGS. 1, 7C, 8C) and/or the plurality of commissures of the replacement heart valve implant 100 (e.g., FIGS. 1, 7C, 8C) are rotationally aligned with the native valve commissures 18 (e.g., FIGS. 7C, 8C) of the native heart valve (e.g., the aortic valve 12). Alignment of the plurality of posts 122 (e.g., FIGS. 1, 7C, 8C) and/or the plurality of commissures of the replacement heart valve implant 100 (e.g., FIGS. 1, 7C, 8C) with the native valve commissures 18 (e.g., FIGS. 7C, 8C) of the native heart valve (e.g., the aortic valve 12) is important to avoid obstruction of the left coronary artery 24 (e.g., FIGS. 1, 7C, 8C) and the right coronary artery 25 (e.g., FIGS. 1, 7C, 8C) with the plurality of posts 122 (e.g., FIGS. 1, 7C, 8C) and/or the plurality of commissures, as the distance between the coronary arteries and the native valve annulus may vary from patient to patient. Furthermore, alignment of the plurality of posts 122 (e.g., FIGS. 1, 7C, 8C) and/or the plurality of commissures of the replacement heart valve implant 100 (e.g., FIGS. 1, 7C, 8C) with the native valve commissures 18 (e.g., FIGS. 7C, 8C) of the native heart valve (e.g., the aortic valve 12) may be useful in maintaining proper valve function and/or limiting, reducing, and/or eliminating paravalvular leakage.

In some embodiments, the implant delivery system 30 and/or the implant holding portion may include an atraumatic transition shield 79, as seen in FIG. 4. The atraumatic transition shield 79 may be disposed adjacent the stent holder 70. In some embodiments, the atraumatic transition shield 79 may be disposed between the stent holder 70 and the handle 40. In some embodiments, the atraumatic transition shield 79 may be disposed proximal the stent holder 70. In some embodiments, the atraumatic transition shield 79 may be disposed at and/or adjacent the first end portion 72 of the stent holder 70. In some embodiments, the atraumatic transition shield 79 may axially overlap the first end portion 72 of the stent holder 70. In some embodiments, the atraumatic transition shield 79 may be disposed radially outward of at least a portion of the first end portion 72 of the stent holder 70. In some embodiments, the atraumatic transition shield 79 may be tapered radially inward in the proximal direction and/or toward the handle 40. The atraumatic transition shield 79 may be configured to prevent the replacement heart valve implant 100, the expandable framework 110, the plurality of valve leaflets 120, etc. (not shown in FIG. 4) from catching on the stent holder 70 as the implant delivery system 30 is withdrawn after deploying the replacement heart valve implant 100.

In use, after advancing and/or navigating the implant delivery system 30 and/or the implant holding portion 60 to the treatment site (over a guidewire, for example), the proximal sheath 62 and/or the distal sheath 64 may be axially translated relative to each other to shift the implant holding portion 60 to the open configuration. When unconstrained by the implant holding portion 60, the replacement heart valve implant 100 (e.g., FIG. 1) and/or the expandable framework 110 (e.g., FIG. 1) may be configured to shift from the radially collapsed configuration (e.g., FIG. 3) to the radially expanded configuration (e.g., FIG. 1). Shifting the replacement heart valve implant 100 (e.g., FIG. 1) and/or the expandable framework 110 toward the radially expanded configuration after axially translating the proximal sheath 62 and/or the distal sheath 64 away from each other and/or the stent holder 70 may permit the replacement heart valve implant 100 (e.g., FIG. 1) and/or the expandable framework 110 to decouple and/or detach from the implant delivery system 30. Some suitable but non-limiting materials for the implant delivery system 30, the handle 40, the elongate shaft assembly 50, and/or components or elements thereof, for example metallic materials and/or polymeric materials, are described below.

In at least some interventions, the replacement heart valve implant 100 (e.g., FIG. 1) may be deployed within the native heart valve (e.g., the native heart valve is left in place). Alternatively, the replacement heart valve implant 100 (e.g., FIG. 1) may be deployed within a previously replaced native heart valve (such as via a surgical aortic valve replacement procedure or via a transcatheter aortic valve replacement procedure) and the replacement heart valve implant 100 may be deployed therein as a replacement.

FIG. 6 illustrates an example configuration of the plurality of longitudinal marker elements 80, along with the stent holder 70, the distal cap 77, and the cage 78 in an exploded view. In some embodiments, the plurality of longitudinal marker elements 80 may comprise a plurality of individual rods 88. In some embodiments, the plurality of individual rods 88 may be formed as solid rods, bars, etc. In some alternative embodiments, the plurality of individual rods 88 may be formed as tubular or hollow rods, bars, etc. In some embodiments, the plurality of individual rods 88 may have a cylindrical shape, a cuboid shape, a rectangular, triangular, or other prismatic shape, a pyramidal shape, etc. In some embodiments, the plurality of individual rods 88 may comprise exactly three individual rods. The plurality of individual rods 88 may be disposed within the distal cap 77.

In some embodiments, the distal cap 77 may comprise a plurality of cavities disposed therein, wherein each cavity of the plurality of cavities is configured to receive one individual rod of the plurality of individual rods 88. In some alternative embodiments, the plurality of individual rods 88 may be embedded within the distal cap 77. For example, the plurality of individual rods 88 may be embedded within a side wall of the distal cap 77 radially inward of the radially outward facing surface 87. In some embodiments, the plurality of individual rods 88 may be disposed radially outward of the intermediate tubular member 56 (not shown in FIG. 6). In some embodiments, the plurality of individual rods 88 may be disposed radially outward of the body 74 of the stent holder 70. Other configurations are also contemplated.

In use, the plurality of individual rods 88 would be visible under fluoroscopy as similar to the plurality of longitudinal marker elements 80 shown in FIGS. 7A-8C, which are discussed in more detail below. When using the plurality of individual rods 88, which may be understood as being or being in place of the plurality of longitudinal marker elements 80 in FIGS. 7A-8C, both the 3-cusp view and the cusp overlap view discussed below are usually required to ensure proper placement of the replacement heart valve implant 100 (e.g., FIGS. 7C, 8C) within the native heart valve unless additional features and/or methods are used, which may increase complexity and/or cost.

When delivering the replacement heart valve implant 100 to the native heart valve (e.g., the aortic valve 12), fluoroscopy is used to visualize the procedure and is well known in the art. In some procedures, an imaging device may be configured to use two different views—the 3-cusp view and the cusp overlap view—and those views would be understood by the skilled artisan. In some embodiments, the 3-cusp view is used initially, and when a satisfactory positioning is seen in the 3-cusp view, the imaging device is switched to the cusp overlap view to verify the orientation and/or positioning of the replacement heart valve implant 100, the plurality of posts 122, and the plurality of commissures with respect to the native heart valve (e.g., the aortic valve 12) and the native valve commissures 18. In some embodiments, the imaging device may be switched between the 3-cusp view and the cusp overlap view multiple times during a procedure.

FIGS. 7A-8C schematically illustrate selected aspects related to using the imaging device to visualize the orientation of the replacement heart valve implant 100, the plurality of posts 122, and the plurality of commissures with respect to the native heart valve (e.g., the aortic valve 12) and the native valve commissures 18.

The 3-cusp view is illustrated schematically in FIGS. 7A and 8A, and the approximate viewpoint for the 3-cusp view is shown in FIG. 7C using the arrow 7A and in FIG. 8C using the arrow 8A. The cusp overlap view is illustrated schematically in FIGS. 7B and 8B, and the approximate viewpoint for the cusp overlap view is shown in FIG. 7C using the arrow 7B and in FIG. 8C using the arrow 8B.

FIGS. 7C and 8C schematically illustrate selected aspects of the replacement heart valve implant 100 and the implant delivery system 30 within the partial cross-sectional view of the native heart valve (e.g., the aortic valve 12) shown in FIG. 2. For clarity, some features of the implant delivery system 30 and the replacement heart valve implant 100 are not shown. Additionally, it should be noted that some illustrated features, such as the plurality of longitudinal marker elements 80, may not actually be visible in the illustrated view, but are shown schematically for reference. As in FIG. 2, the left coronary cusp L, the right coronary cusp R, and the non-coronary cusp N are shown in FIGS. 7C and 8C. FIGS. 7C and 8C illustrate two example orientations of the replacement heart valve implant 100, the plurality of posts 122, and the plurality of commissures with respect to the native heart valve (e.g., the aortic valve 12) and the native valve commissures 18 that illustrate the benefit of using both the 3-cusp view and the cusp overlap view.

As may be seen when comparing FIGS. 7A and 8A to FIGS. 7C and 8C, the plurality of longitudinal marker elements 80 may be seen generally aligned with the native valve commissures 18 in the 3-cusp view. However, as shown in FIGS. 7C and 8C, the replacement heart valve implant 100 may be in one of two different orientations relative to the native heart valve and/or the native valve commissures 18. This may be caused by inherent limitations of the 2-dimensional nature of the 3-cusp view when viewing the 3-dimensional arrangement of the plurality of longitudinal marker elements 80. In FIG. 7C, the plurality of longitudinal marker elements 80 and the plurality of posts 122 (and the plurality of commissures) of the replacement heart valve implant 100 are rotationally aligned with the native valve commissures 18, while in FIG. 8C, the plurality of longitudinal marker elements 80 and the plurality of posts 122 (and the plurality of commissures) of the replacement heart valve implant 100 are rotationally misaligned with the native valve commissures 18, even though the 3-cusp views (e.g., FIG. 7A and FIG. 8A) corresponding to each of the arrangements in FIGS. 7C and 8C look the same (e.g., the plurality of longitudinal marker elements 80 appear to be substantially equally spaced apart). Rotational misalignment of the plurality of posts 122 (and the plurality of commissures) of the replacement heart valve implant 100 with respect to the native valve commissures 18 may cause the plurality of posts 122 (and the plurality of commissures) of the replacement heart valve implant 100 to at least partially overlap the left coronary artery 24 and the right coronary artery 25, as seen in FIG. 8C. A further complication known as the parallax effect is also possible. The parallax effect is characterized by the position of an object or a feature appearing different when viewed (e.g., imaged and/or visualized) from different positions. Parallax, in the context of implanting the replacement heart valve implant 100, may result in the replacement heart valve implant 100 being positioned incorrectly (e.g., too deep or not deep enough relative to the native valve annulus). The use of multiple views (e.g., the 3-cusp view and the cusp overlap view) may permit the practitioner to identify parallax and correct for it.

Accordingly, once the plurality of longitudinal marker elements 80 are seen generally aligned with the native valve commissures 18 in the 3-cusp view, as shown in FIGS. 7A and 8A, the imaging device may be switched to the cusp overlap view shown in FIGS. 7B and 8B. From this viewpoint, the left coronary cusp L and the right coronary cusp R may be seen to substantially overlap in the right-hand portion of the view, with the non-coronary cusp N left alone in the left-hand portion of the view. This arrangement may be used to visualize the positioning of the plurality of longitudinal marker elements 80 with respect to the left coronary cusp L, the right coronary cusp R, and the non-coronary cusp N.

If the replacement heart valve implant 100 is positioned correctly (e.g., a desired final orientation), as in FIG. 7C, with the plurality of longitudinal marker elements 80 and the plurality of posts 122 (and the plurality of commissures) of the replacement heart valve implant 100 rotationally aligned with the native valve commissures 18, the replacement heart valve implant 100 will be seen as having a single longitudinal marker element of the plurality of longitudinal marker elements 80 positioned farther away from the non-coronary cusp N of the native heart valve (e.g., the aortic valve 12) than other marker elements of the plurality of longitudinal marker elements 80 in the cusp overlap view, as shown in FIG. 7B. For example, in the cusp overlap view of FIG. 7B, if the replacement heart valve implant 100 is positioned correctly, a single longitudinal marker element of the plurality of longitudinal marker elements 80 is positioned to the right of two longitudinal marker elements of the plurality of longitudinal marker elements 80, which may be overlapping and/or in very close proximity to each other.

If the replacement heart valve implant 100 is positioned incorrectly, as in FIG. 8C, with the plurality of longitudinal marker elements 80 and the plurality of posts 122 (and the plurality of commissures) of the replacement heart valve implant 100 rotationally misaligned with the native valve commissures 18, the replacement heart valve implant 100 will be seen as having a single longitudinal marker element of the plurality of longitudinal marker elements 80 positioned closer to the non-coronary cusp N of the native heart valve (e.g., the aortic valve 12) than other marker elements of the plurality of longitudinal marker elements 80 in the cusp overlap view, as shown in FIG. 8B. For example, in the cusp overlap view of FIG. 8B, if the replacement heart valve implant 100 is positioned incorrectly, a single longitudinal marker element of the plurality of longitudinal marker elements 80 is positioned to the left of two longitudinal marker elements of the plurality of longitudinal marker elements 80, which may be overlapping and/or in very close proximity to each other.

Accordingly, the relative position of a single longitudinal marker element of the plurality of longitudinal marker elements 80 with respect to other marker elements of the plurality of longitudinal marker elements 80 in the cusp overlap view (e.g., FIGS. 7B and 8B) may be useful to determine whether the plurality of posts 122 and the plurality of commissures of the replacement heart valve implant 100 overlap the left coronary artery 24 and the right coronary artery 25. A practitioner may use both the 3-cusp view and the cusp overlap view when delivering the replacement heart valve implant 100 to ensure proper placement and/or orientation within the native heart valve (e.g., the aortic valve 12).

During delivery of the replacement heart valve implant 100 to the native heart valve, deployment of the replacement heart valve implant 100 may include shifting the replacement heart valve implant 100 from the radially collapsed configuration to the radially expanded configuration. In some embodiments, the proximal portion of the replacement heart valve implant 100 may be released and/or radially expanded first and/or before the distal portion of the replacement heart valve implant 100.

As discussed herein, the plurality of longitudinal marker elements 80 may be aligned with the plurality of posts 122 and the plurality of commissures of the replacement heart valve implant 100 when the replacement heart valve implant 100 is constrained within the implant holding portion 60 of the implant delivery system 30. The plurality of longitudinal marker elements 80 may be configured to rotationally align commissures of the replacement heart valve implant 100 with native valve commissures 18 of the native heart valve (e.g., the aortic valve 12) under fluoroscopy with the imaging device when the replacement heart valve implant 100 is shifted from the radially collapsed configuration to the radially expanded configuration within the native heart valve (e.g., the aortic valve 12). Accordingly, if the plurality of longitudinal marker elements 80 is rotationally aligned with the native valve commissures 18 of the native heart valve (e.g., the aortic valve 12) when the replacement heart valve implant 100 is shifted from the radially collapsed configuration to the radially expanded configuration within the native heart valve (e.g., the aortic valve 12), the plurality of posts 122 and the plurality of commissures of the replacement heart valve implant 100 will be aligned with the native valve commissures 18 of the native heart valve (e.g., the aortic valve 12).

The applicants have identified features and/or ways to improve imaging of the implant delivery system 30 in situ to identify parallax and/or to reduce the number of views needed and/or used under fluoroscopy, thereby speeding up the procedure while maintaining and/or improving patient safety and procedural outcome.

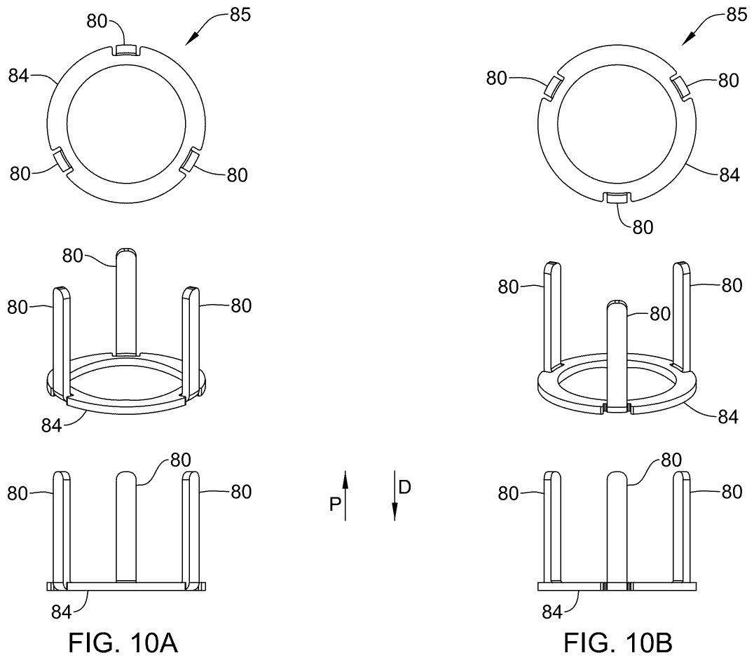

FIGS. 9-18B illustrate example configurations of the plurality of longitudinal marker elements 80, the annular ring 84, and/or the commissure marker 85 according to the disclosure, along with the stent holder 70, the distal cap 77, and the cage 78 in exploded and detailed views. As seen in FIGS. 9, 11, 13, 15, and 17, different orientations for the plurality of longitudinal marker elements 80, the annular ring 84, and/or the commissure marker 85 are contemplated. FIGS. 10A-10B, 12A-12B, 14A-14B, 16A-16B, and 18A-18B illustrate several views of the commissure marker 85 of FIGS. 9, 11, 13, 15, and 17, respectively, including a view from proximal to distal, a tilted view, and a side view. In each of these figures, a proximal direction P and a distal direction D are identified in the side view with arrows corresponding to proximal and distal orientations within the implant delivery system 30 (e.g., FIG. 3).

One aspect of positioning the replacement heart valve implant 100 (e.g., FIGS. 7C, 8C) that may be useful is that if the replacement heart valve implant 100 is positioned correctly (e.g., the desired final orientation), as in FIG. 7C, a middle longitudinal marker element of the plurality of longitudinal marker elements 80 is disposed in a posterior position relative to the imaging device in the 3-cusp view, and if the replacement heart valve implant 100 is positioned incorrectly, as in FIG. 8C, the middle longitudinal marker element of the plurality of longitudinal marker elements 80 is disposed in an anterior position relative to the imaging device in the 3-cusp view. Accordingly, identifying the location and/or position of the middle longitudinal marker element of the plurality of longitudinal marker elements 80 in the 3-cusp view may be useful for streamlining the procedure while maintaining and/or improving patient safety and procedural outcome.

FIGS. 9, 10A, and 10B illustrate the commissure marker 85 and/or the plurality of longitudinal marker elements 80 with the annular ring 84 disposed in a distal orientation and/or spaced distally away from the stent holder 70. The plurality of longitudinal marker elements 80 extends proximally from the annular ring 84. The plurality of longitudinal marker elements 80 may be disposed within the distal cap 77. In some embodiments, the distal cap 77 may comprise a plurality of cavities disposed therein, wherein each cavity of the plurality of cavities is configured to receive one longitudinal marker element of the plurality of longitudinal marker elements 80. In some alternative embodiments, the plurality of longitudinal marker elements 80 and/or the annular ring 84 may be at least partially embedded within the distal cap 77. For example, the plurality of longitudinal marker elements 80 may be embedded within a side wall of the distal cap 77 radially inward of the radially outward facing surface 87. In some embodiments, the plurality of longitudinal marker elements 80 may be completely embedded within the side wall of the distal cap 77. In some embodiments, the annular ring 84 may be at least partially embedded within the distal cap 77. In some embodiments, a distal face or a portion of the distal face of the annular ring 84 may be exposed from the distal cap 77. In some embodiments, the distal cap 77 may be co-molded, extruded, injection molded, etc. with and/or around the commissure marker 85.

In some embodiments, the annular ring 84 may have a thickness measured in a longitudinal direction (e.g., proximal to distal) of about 0.070 millimeters (mm) to about 0.225 mm, about 0.075 mm to about 0.200 mm, about 0.080 to about 0.175 mm, etc. Other configurations are also contemplated.

In some embodiments, the plurality of longitudinal marker elements 80 may be disposed radially outward of the intermediate tubular member 56 (not shown in FIG. 9). In some embodiments, the plurality of longitudinal marker elements 80 may be disposed radially outward of the body 74 of the stent holder 70. Other configurations are also contemplated. In use, the plurality of longitudinal marker elements 80 and the annular ring 84 would be visible under fluoroscopy, similar to FIGS. 7A-8C above. In the 3-cusp view, the commissure marker 85, the annular ring 84, and/or the plurality of longitudinal marker elements 80 would generally appear as seen in the side views of FIGS. 10A-10B, wherein FIG. 10A corresponds to the arrangement of FIG. 7A and FIG. 10B corresponds to the arrangement of FIG. 8A.

For reference, while the commissure marker 85 and the annular ring 84 are shown in the tilted views of FIGS. 10A-10B as being tilted in a forward direction (e.g., an anterior portion of the annular ring 84 is lower or more distal than a posterior portion of the annular ring 84) relative to the imaging device in the 3-cusp view, the skilled person will recognize that similar views (e.g., the annular ring 84 appearing as an oval in the 3-cusp view) may be obtained if the commissure marker 85 and the annular ring 84 are tilted in a backward direction (e.g., the posterior portion of the annular ring 84 is lower or more distal than the anterior portion of the annular ring 84) relative to the imaging device in the 3-cusp view. Any tilt in either direction could be considered to be representative of parallax.

In some embodiments, when using the commissure marker 85 with the annular ring 84 and the plurality of longitudinal marker elements 80 shown in FIG. 9, the replacement heart valve implant 100 (e.g., FIGS. 7C, 8C) may be properly placed within the native heart valve with respect to the native valve commissures 18 (e.g., FIGS. 7C, 8C) using only the 3-cusp view. In one example, the annular ring 84 may be visible as an oval under fluoroscopy when the implant delivery system 30 (e.g., FIG. 3) is tilted toward or away from the imaging device (e.g., the tilted views of FIGS. 10A and 10B), and the location of the middle longitudinal marker element of the plurality of longitudinal marker elements 80 may be visible under fluoroscopy (e.g., the middle longitudinal marker element may be visible in the anterior position or the posterior position based on its intersection with the annular ring 84). When the replacement heart valve implant 100 (e.g., FIGS. 7C, 8C) is positioned properly within the native heart valve with respect to the native valve commissures 18 (e.g., FIGS. 7C, 8C), the middle longitudinal marker element will be disposed in the posterior position.

In some embodiments, when using the commissure marker 85 with the annular ring 84 and the plurality of longitudinal marker elements 80 shown in FIG. 9, both the 3-cusp view and the cusp overlap view discussed herein may be required to ensure proper placement of the replacement heart valve implant 100 (e.g., FIGS. 7C, 8C) within the native heart valve with respect to the native valve commissures 18 (e.g., FIGS. 7C, 8C). However, the annular ring 84 may provide the benefit of being able to identify parallax via visualization of forward or backward tilting of the implant delivery system 30 and/or the replacement heart valve implant 100 with respect to the imaging device in the 3-cusp view by visualizing the annular ring 84 under fluoroscopy as an oval in the 3-cusp view instead of a substantially straight line. When the replacement heart valve implant 100 (e.g., FIGS. 7C, 8C) is positioned properly within the native heart valve with no parallax (e.g., no forward or backward tilt with respect to the imaging device in the 3-cusp view), the annular ring 84 will appear under fluoroscopy as a substantially straight line in the 3-cusp view (e.g., the side views of FIGS. 10A-10B). Accordingly, parallax may be identified without changing to the cusp overlap view and/or using only the 3-cusp view.

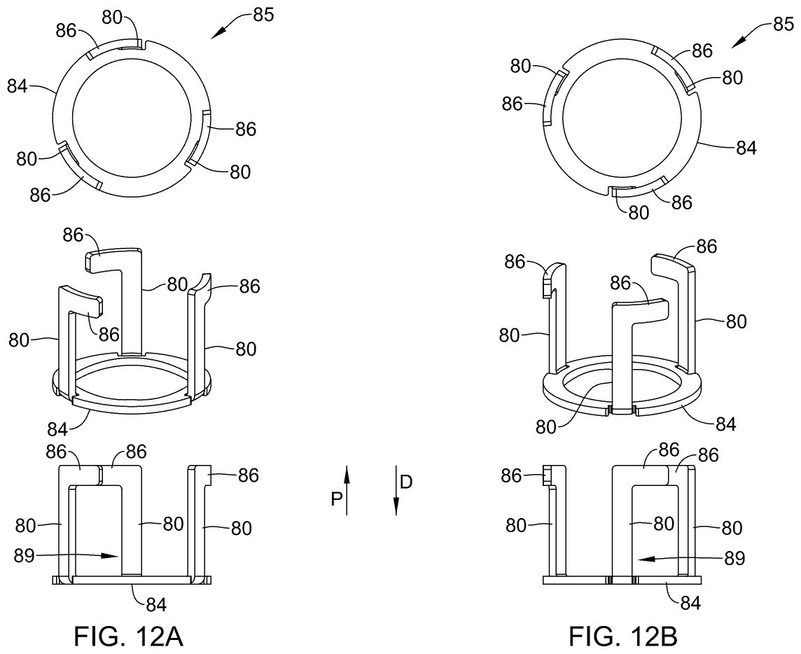

FIGS. 11, 12A, and 12B illustrate the commissure marker 85 and/or the plurality of longitudinal marker elements 80 with the annular ring 84 disposed in a distal orientation and/or spaced distally away from the stent holder 70. The plurality of longitudinal marker elements 80 extends proximally from the annular ring 84. Each longitudinal marker element of the plurality of longitudinal marker elements 80 comprises the non-longitudinal directional marker element 86. Each non-longitudinal directional marker element 86 is shown extending to the left and/or counterclockwise from its respective longitudinal marker element as viewed in a proximal to distal direction (e.g., from right to left in FIG. 11) along the central longitudinal axis of the elongate shaft assembly 50 (not shown in FIG. 11).

The plurality of longitudinal marker elements 80 may be disposed within the distal cap 77. Similarly, each non-longitudinal directional marker element 86 of the plurality of longitudinal marker elements 80 may be disposed within the distal cap 77. In some embodiments, the distal cap 77 may comprise a plurality of cavities disposed therein, wherein each cavity of the plurality of cavities is configured to receive one longitudinal marker element of the plurality of longitudinal marker elements 80 and/or its respective non-longitudinal directional marker element 86. In some alternative embodiments, the plurality of longitudinal marker elements 80, each non-longitudinal directional marker element 86 of the plurality of longitudinal marker elements 80, and/or the annular ring 84 may be at least partially embedded within the distal cap 77. For example, the plurality of longitudinal marker elements 80 and/or each non-longitudinal directional marker element 86 of the plurality of longitudinal marker elements 80 may be embedded within a side wall of the distal cap 77 radially inward of the radially outward facing surface 87. In some embodiments, the plurality of longitudinal marker elements 80 and/or each non-longitudinal directional marker element 86 of the plurality of longitudinal marker elements 80 may be completely embedded within the side wall of the distal cap 77. In some embodiments, the annular ring 84 may be at least partially embedded within the distal cap 77. In some embodiments, a distal face or a portion of the distal face of the annular ring 84 may be exposed from the distal cap 77. In some embodiments, the distal cap 77 may be co-molded, extruded, injection molded, etc. with and/or around the commissure marker 85.

In some embodiments, the plurality of longitudinal marker elements 80 may have a length in the longitudinal direction (e.g., proximal to distal) that is about 2 times, about 3 times, about 4 times, or about 5 times greater than a length of its corresponding non-longitudinal directional marker element 86 measured in a lateral or circumferential direction (e.g., generally perpendicular to the longitudinal direction in a side view). The difference in lengths may be useful for providing visual distinction of direction and/or orientation of the features. In some embodiments, each non-longitudinal directional marker element 86 and/or the annular ring 84 may have a thickness measured in a longitudinal direction (e.g., proximal to distal) of about 0.070 millimeters (mm) to about 0.225 mm, about 0.075 mm to about 0.200 mm, about 0.080 to about 0.175 mm, etc. Other configurations are also contemplated.

In some embodiments, the plurality of longitudinal marker elements 80 and/or each non-longitudinal directional marker element 86 of the plurality of longitudinal marker elements 80 may be disposed radially outward of the intermediate tubular member 56 (not shown in FIG. 11). In some embodiments, the plurality of longitudinal marker elements 80 and/or each non-longitudinal directional marker element 86 of the plurality of longitudinal marker elements 80 may be disposed radially outward of the body 74 of the stent holder 70. Other configurations are also contemplated. In use, the plurality of longitudinal marker elements 80, each non-longitudinal directional marker element 86 of the plurality of longitudinal marker elements 80, and the annular ring 84 would be visible under fluoroscopy, similar to FIGS. 7A-8C above. In the 3-cusp view, the commissure marker 85, the annular ring 84, and/or the plurality of longitudinal marker elements 80 would generally appear as seen in the side views of FIGS. 12A-12B, wherein FIG. 12A corresponds to the arrangement of FIG. 7A and FIG. 12B corresponds to the arrangement of FIG. 8A.

For reference, while the commissure marker 85 and the annular ring 84 are shown in the tilted views of FIGS. 12A-12B as being tilted in a forward direction (e.g., an anterior portion of the annular ring 84 is lower or more distal than a posterior portion of the annular ring 84) relative to the imaging device in the 3-cusp view, the skilled person will recognize that similar views (e.g., the annular ring 84 appearing as an oval in the 3-cusp view) may be obtained if the commissure marker 85 and the annular ring 84 are tilted in a backward direction (e.g., the posterior portion of the annular ring 84 is lower or more distal than the anterior portion of the annular ring 84) relative to the imaging device in the 3-cusp view. Any tilt in either direction could be considered to be representative of parallax.

In some embodiments, when using the commissure marker 85 with the annular ring 84, the plurality of longitudinal marker elements 80, and each non-longitudinal directional marker element 86 of the plurality of longitudinal marker elements 80 shown in FIG. 11, the replacement heart valve implant 100 (e.g., FIGS. 7C, 8C) may be properly placed within the native heart valve with respect to the native valve commissures 18 (e.g., FIGS. 7C, 8C) using only the 3-cusp view. In one example, each non-longitudinal directional marker element 86 of the plurality of longitudinal marker elements 80 may be visible under fluoroscopy in the 3-cusp view.

The direction the non-longitudinal directional marker element 86 of the middle longitudinal marker element extends may be used to identify whether the middle longitudinal marker element is disposed in the posterior position or the anterior position. For example, when the non-longitudinal directional marker element 86 of the middle longitudinal marker element extends to the left in the 3-cusp view (e.g., the side view of FIG. 12A), the middle longitudinal marker element may be disposed in the posterior position, as seen in FIG. 12A, and when the non-longitudinal directional marker element 86 of the middle longitudinal marker element extends to the right in the 3-cusp view (e.g., the side view of FIG. 12B), the middle longitudinal marker element may be disposed in the anterior position, as seen in FIG. 12B. In some alternative embodiments, these directions may be reversed, and the practitioner would be aware of the changes/differences before the procedure.

In some embodiments, the position of the middle longitudinal marker element may be determined via a configuration formed by the commissure marker 85, and/or a combination of the plurality of longitudinal marker elements 80, their non-longitudinal directional marker elements (ref. 86), and the annular ring 84. For example, in some embodiments, the annular ring 84, the plurality of longitudinal marker elements 80, and each non-longitudinal directional marker element 86 of the plurality of longitudinal marker elements 80 may form a box 89, a closed geometric feature, etc. under fluoroscopy in the 3-cusp view (e.g., the side views of FIGS. 12A-12B) such that there is no gap between two longitudinal marker elements of the plurality of longitudinal marker elements 80. When the box 89, or no gap between two longitudinal marker elements of the plurality of longitudinal marker elements 80, is disposed to the left of the middle longitudinal marker element, under fluoroscopy in the 3-cusp view (e.g., the side view of FIG. 12A), the middle longitudinal marker element may be disposed in the posterior position, and when the box 89, or no gap between two longitudinal marker elements of the plurality of longitudinal marker elements 80, is disposed to the right of the middle longitudinal marker element under fluoroscopy in the 3-cusp view (e.g., the side view of FIG. 12B), the middle longitudinal marker element may be disposed in the anterior position. In some alternative embodiments, these directions may be reversed, and the practitioner would be aware of the changes/differences before the procedure.

Accordingly, in at least some embodiments, the position of the middle longitudinal marker element may be determined without changing from the 3-cusp view. When the replacement heart valve implant 100 (e.g., FIGS. 7C, 8C) is positioned properly within the native heart valve with respect to the native valve commissures 18 (e.g., FIGS. 7C, 8C), the middle longitudinal marker element will be disposed in the posterior position.

Additionally, the annular ring 84 may provide the benefit of being able to identify parallax via visualization of forward or backward tilting of the implant delivery system 30 and/or the replacement heart valve implant 100 with respect to the imaging device in the 3-cusp view by visualizing the annular ring 84 under fluoroscopy as an oval in the 3-cusp view (e.g., the tilted views of FIGS. 12A-12B) instead of a substantially straight line. When the replacement heart valve implant 100 (e.g., FIGS. 7C, 8C) is positioned properly within the native heart valve with no parallax (e.g., no forward or backward tilt with respect to the imaging device), the annular ring 84 will appear under fluoroscopy as a substantially straight line in the 3-cusp view (e.g., the side views of FIGS. 12A-12B). Accordingly, parallax may be identified without changing to the cusp overlap view and/or using only the 3-cusp view.

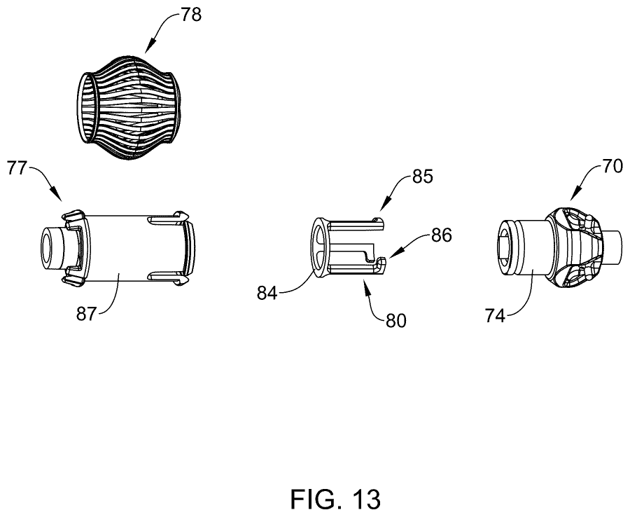

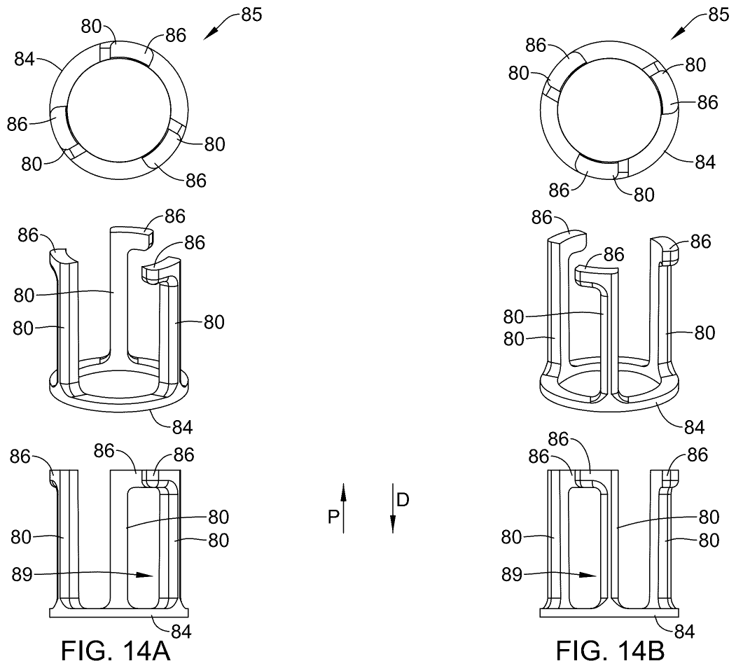

FIGS. 13, 14A, and 14B illustrate the commissure marker 85 and/or the plurality of longitudinal marker elements 80 with the annular ring 84 disposed in a distal orientation and/or spaced distally away from the stent holder 70. The plurality of longitudinal marker elements 80 extends proximally from the annular ring 84. Each longitudinal marker element of the plurality of longitudinal marker elements 80 comprises the non-longitudinal directional marker element 86. Each non-longitudinal directional marker element 86 is shown extending to the right and/or clockwise from its respective longitudinal marker element as viewed in a proximal to distal direction (e.g., from right to left in FIG. 13) along the central longitudinal axis of the elongate shaft assembly 50 (not shown in FIG. 13).

The plurality of longitudinal marker elements 80 may be disposed within the distal cap 77. Similarly, each non-longitudinal directional marker element 86 of the plurality of longitudinal marker elements 80 may be disposed within the distal cap 77. In some embodiments, the distal cap 77 may comprise a plurality of cavities disposed therein, wherein each cavity of the plurality of cavities is configured to receive one longitudinal marker element of the plurality of longitudinal marker elements 80 and/or its respective non-longitudinal directional marker element 86. In some alternative embodiments, the plurality of longitudinal marker elements 80, each non-longitudinal directional marker element 86 of the plurality of longitudinal marker elements 80, and/or the annular ring 84 may be at least partially embedded within the distal cap 77. For example, the plurality of longitudinal marker elements 80 and/or each non-longitudinal directional marker element 86 of the plurality of longitudinal marker elements 80 may be embedded within a side wall of the distal cap 77 radially inward of the radially outward facing surface 87. In some embodiments, the plurality of longitudinal marker elements 80 and/or each non-longitudinal directional marker element 86 of the plurality of longitudinal marker elements 80 may be completely embedded within the side wall of the distal cap 77. In some embodiments, the annular ring 84 may be at least partially embedded within the distal cap 77. In some embodiments, a distal face or a portion of the distal face of the annular ring 84 may be exposed from the distal cap 77. In some embodiments, the distal cap 77 may be co-molded, extruded, injection molded, etc. with and/or around the commissure marker 85.

In some embodiments, the plurality of longitudinal marker elements 80 may have a length in the longitudinal direction (e.g., proximal to distal) that is about 2 times, about 3 times, about 4 times, or about 5 times greater than a length of its corresponding non-longitudinal directional marker element 86 measured in a lateral or circumferential direction (e.g., generally perpendicular to the longitudinal direction in a side view). The difference in lengths may be useful for providing visual distinction of direction and/or orientation of the features. In some embodiments, each non-longitudinal directional marker element 86 and/or the annular ring 84 may have a thickness measured in a longitudinal direction (e.g., proximal to distal) of about 0.070 millimeters (mm) to about 0.225 mm, about 0.075 mm to about 0.200 mm, about 0.080 to about 0.175 mm, etc. Other configurations are also contemplated.

In some embodiments, the plurality of longitudinal marker elements 80 and/or each non-longitudinal directional marker element 86 of the plurality of longitudinal marker elements 80 may be disposed radially outward of the intermediate tubular member 56 (not shown in FIG. 13). In some embodiments, the plurality of longitudinal marker elements 80 and/or each non-longitudinal directional marker element 86 of the plurality of longitudinal marker elements 80 may be disposed radially outward of the body 74 of the stent holder 70. Other configurations are also contemplated. In use, the plurality of longitudinal marker elements 80, each non-longitudinal directional marker element 86 of the plurality of longitudinal marker elements 80, and the annular ring 84 would be visible under fluoroscopy, similar to FIGS. 7A-8C above. In the 3-cusp view, the commissure marker 85, the annular ring 84, and/or the plurality of longitudinal marker elements 80 would generally appear as seen in the side views of FIGS. 14A-14B, wherein FIG. 14A corresponds to the arrangement of FIG. 7A and FIG. 14B corresponds to the arrangement of FIG. 8A.

For reference, while the commissure marker 85 and the annular ring 84 are shown in the tilted views of FIGS. 14A-14B as being tilted in a forward direction (e.g., an anterior portion of the annular ring 84 is lower or more distal than a posterior portion of the annular ring 84) relative to the imaging device in the 3-cusp view, the skilled person will recognize that similar views (e.g., the annular ring 84 appearing as an oval in the 3-cusp view) may be obtained if the commissure marker 85 and the annular ring 84 are tilted in a backward direction (e.g., the posterior portion of the annular ring 84 is lower or more distal than the anterior portion of the annular ring 84) relative to the imaging device in the 3-cusp view. Any tilt in either direction could be considered to be representative of parallax.

In some embodiments, when using the commissure marker 85 with the annular ring 84, the plurality of longitudinal marker elements 80, and each non-longitudinal directional marker element 86 of the plurality of longitudinal marker elements 80 shown in FIG. 13, the replacement heart valve implant 100 (e.g., FIGS. 7C, 8C) may be properly placed within the native heart valve with respect to the native valve commissures 18 (e.g., FIGS. 7C, 8C) using only the 3-cusp view. In one example, each non-longitudinal directional marker element 86 of the plurality of longitudinal marker elements 80 may be visible under fluoroscopy in the 3-cusp view.

The direction the non-longitudinal directional marker element 86 of the middle longitudinal marker element extends may be used to identify whether the middle longitudinal marker element is disposed in the posterior position or the anterior position. For example, when the non-longitudinal directional marker element 86 of the middle longitudinal marker element extends to the right in the 3-cusp view (e.g., the side view of FIG. 14A), the middle longitudinal marker element may be disposed in the posterior position, as seen in FIG. 14A, and when the non-longitudinal directional marker element 86 of the middle longitudinal marker element extends to the left in the 3-cusp view (e.g., the side view of FIG. 14B), the middle longitudinal marker element may be disposed in the anterior position, as seen in FIG. 14B. In some alternative embodiments, these directions may be reversed, and the practitioner would be aware of the changes/differences before the procedure.

In some embodiments, the position of the middle longitudinal marker element may be determined via a configuration formed by the commissure marker 85, and/or a combination of the plurality of longitudinal marker elements 80, their non-longitudinal directional marker elements (ref. 86), and the annular ring 84. For example, in some embodiments, the annular ring 84, the plurality of longitudinal marker elements 80, and each non-longitudinal directional marker element 86 of the plurality of longitudinal marker elements 80 may form a box 89, a closed geometric feature, etc. under fluoroscopy in the 3-cusp view (e.g., the side views of FIGS. 14A-14B) such that there is no gap between two longitudinal marker elements of the plurality of longitudinal marker elements 80. When the box 89, or no gap between two longitudinal marker elements of the plurality of longitudinal marker elements 80, is disposed to the right of the middle longitudinal marker element, under fluoroscopy in the 3-cusp view (e.g., the side view of FIG. 14A), the middle longitudinal marker element may be disposed in the posterior position, and when the box 89, or no gap between two longitudinal marker elements of the plurality of longitudinal marker elements 80, is disposed to the left of the middle longitudinal marker element under fluoroscopy in the 3-cusp view (e.g., the side view of FIG. 14B), the middle longitudinal marker element may be disposed in the anterior position. In some alternative embodiments, these directions may be reversed, and the practitioner would be aware of the changes/differences before the procedure.

Accordingly, in at least some embodiments, the position of the middle longitudinal marker element may be determined without changing from the 3-cusp view. When the replacement heart valve implant 100 (e.g., FIGS. 7C, 8C) is positioned properly within the native heart valve with respect to the native valve commissures 18 (e.g., FIGS. 7C, 8C), the middle longitudinal marker element will be disposed in the posterior position.

Additionally, the annular ring 84 may provide the benefit of being able to identify parallax via visualization of forward or backward tilting of the implant delivery system 30 and/or the replacement heart valve implant 100 with respect to the imaging device in the 3-cusp view by visualizing the annular ring 84 under fluoroscopy as an oval in the 3-cusp view (e.g., the tilted views of FIGS. 14A-14B) instead of a substantially straight line. When the replacement heart valve implant 100 (e.g., FIGS. 7C, 8C) is positioned properly within the native heart valve with no parallax (e.g., no forward or backward tilt with respect to the imaging device), the annular ring 84 will appear under fluoroscopy as a substantially straight line in the 3-cusp view (e.g., the side views of FIGS. 14A-14B). Accordingly, parallax may be identified without changing to the cusp overlap view and/or using only the 3-cusp view.

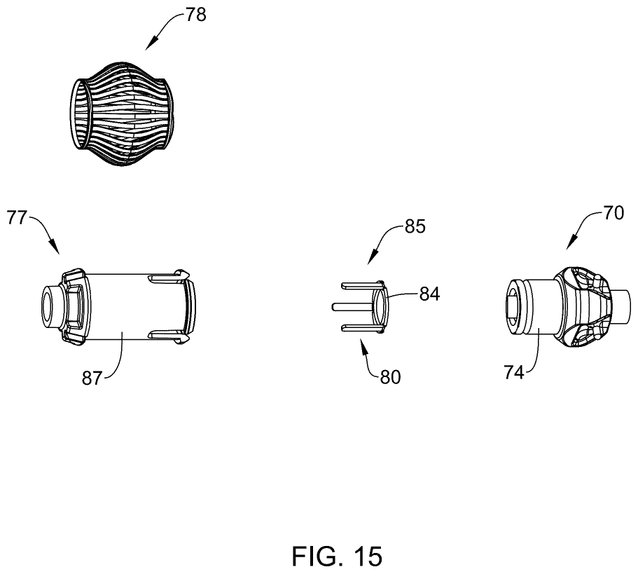

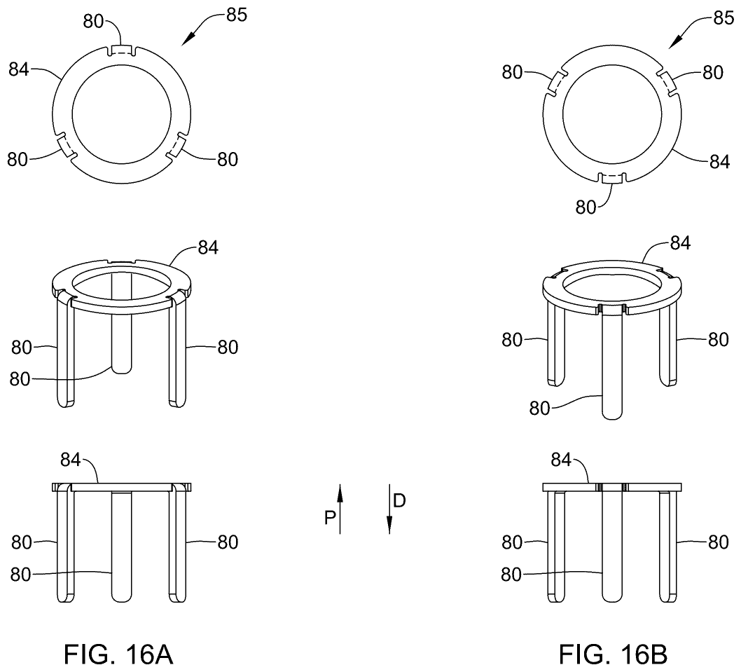

FIGS. 15, 16A, and 16B illustrate the commissure marker 85 and/or the plurality of longitudinal marker elements 80 with the annular ring 84 disposed in a proximal orientation. The plurality of longitudinal marker elements 80 extends distally from the annular ring 84. The plurality of longitudinal marker elements 80 may be disposed within the distal cap 77. In some embodiments, the distal cap 77 may comprise a plurality of cavities disposed therein, wherein each cavity of the plurality of cavities is configured to receive one longitudinal marker element of the plurality of longitudinal marker elements 80. In some alternative embodiments, the plurality of longitudinal marker elements 80 and/or the annular ring 84 may be at least partially embedded within the distal cap 77. For example, the plurality of longitudinal marker elements 80 may be embedded within a side wall of the distal cap 77 radially inward of the radially outward facing surface 87. In some embodiments, the plurality of longitudinal marker elements 80 may be completely embedded within the side wall of the distal cap 77. In some embodiments, the annular ring 84 may be at least partially embedded within the distal cap 77. In some embodiments, a distal face or a portion of the distal face of the annular ring 84 may be exposed from the distal cap 77. In some embodiments, the distal cap 77 may be co-molded, extruded, injection molded, etc. with and/or around the commissure marker 85.

In some embodiments, the annular ring 84 may have a thickness measured in a longitudinal direction (e.g., proximal to distal) of about 0.070 millimeters (mm) to about 0.225 mm, about 0.075 mm to about 0.200 mm, about 0.080 to about 0.175 mm, etc. Other configurations are also contemplated.

In some embodiments, the plurality of longitudinal marker elements 80 may be disposed radially outward of the intermediate tubular member 56 (not shown in FIG. 15). In some embodiments, the plurality of longitudinal marker elements 80 may be disposed radially outward of the body 74 of the stent holder 70. Other configurations are also contemplated. In use, the plurality of longitudinal marker elements 80 and the annular ring 84 would be visible under fluoroscopy, similar to FIGS. 7A-8C above. In the 3-cusp view, the commissure marker 85, the annular ring 84, and/or the plurality of longitudinal marker elements 80 would generally appear as seen in the side views of FIGS. 16A-16B, wherein FIG. 16A corresponds to the arrangement of FIG. 7A and FIG. 16B corresponds to the arrangement of FIG. 8A.

For reference, while the commissure marker 85 and the annular ring 84 are shown in the tilted views of FIGS. 16A-16B as being tilted in a forward direction (e.g., an anterior portion of the annular ring 84 is lower or more distal than a posterior portion of the annular ring 84) relative to the imaging device in the 3-cusp view, the skilled person will recognize that similar views (e.g., the annular ring 84 appearing as an oval in the 3-cusp view) may be obtained if the commissure marker 85 and the annular ring 84 are tilted in a backward direction (e.g., the posterior portion of the annular ring 84 is lower or more distal than the anterior portion of the annular ring 84) relative to the imaging device in the 3-cusp view. Any tilt in either direction could be considered to be representative of parallax.

In some embodiments, when using the commissure marker 85 with the annular ring 84 and the plurality of longitudinal marker elements 80 shown in FIG. 15, the replacement heart valve implant 100 (e.g., FIGS. 7C, 8C) may be properly placed within the native heart valve with respect to the native valve commissures 18 (e.g., FIGS. 7C, 8C) using only the 3-cusp view. In one example, the annular ring 84 may be visible as an oval under fluoroscopy when the implant delivery system 30 (e.g., FIG. 3) is tilted toward or away from the imaging device (e.g., the tilted views of FIGS. 16A and 16B), and the location of the middle longitudinal marker element of the plurality of longitudinal marker elements 80 may be visible under fluoroscopy (e.g., the middle longitudinal marker element may be visible in the anterior position or the posterior position based on its intersection with the annular ring 84). When the replacement heart valve implant 100 (e.g., FIGS. 7C, 8C) is positioned properly within the native heart valve with respect to the native valve commissures 18 (e.g., FIGS. 7C, 8C), the middle longitudinal marker element will be disposed in the posterior position.

In some embodiments, when using the commissure marker 85 with the annular ring 84 and the plurality of longitudinal marker elements 80 shown in FIG. 15, both the 3-cusp view and the cusp overlap view discussed herein may be required to ensure proper placement of the replacement heart valve implant 100 (e.g., FIGS. 7C, 8C) within the native heart valve with respect to the native valve commissures 18 (e.g., FIGS. 7C, 8C). However, the annular ring 84 may provide the benefit of being able to identify parallax via visualization of forward or backward tilting of the implant delivery system 30 and/or the replacement heart valve implant 100 with respect to the imaging device in the 3-cusp view by visualizing the annular ring 84 under fluoroscopy as an oval in the 3-cusp view instead of a substantially straight line. When the replacement heart valve implant 100 (e.g., FIGS. 7C, 8C) is positioned properly within the native heart valve with no parallax (e.g., no forward or backward tilt with respect to the imaging device in the 3-cusp view), the annular ring 84 will appear under fluoroscopy as a substantially straight line in the 3-cusp view (e.g., the side views of FIGS. 16A-16B). Accordingly, parallax may be identified without changing to the cusp overlap view and/or using only the 3-cusp view.

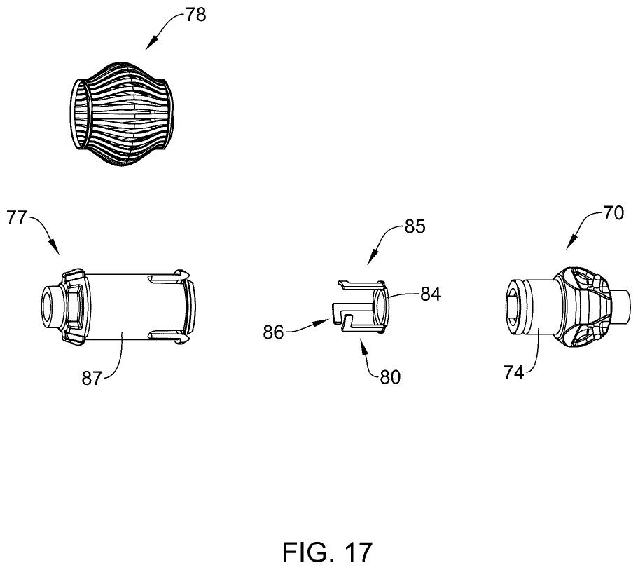

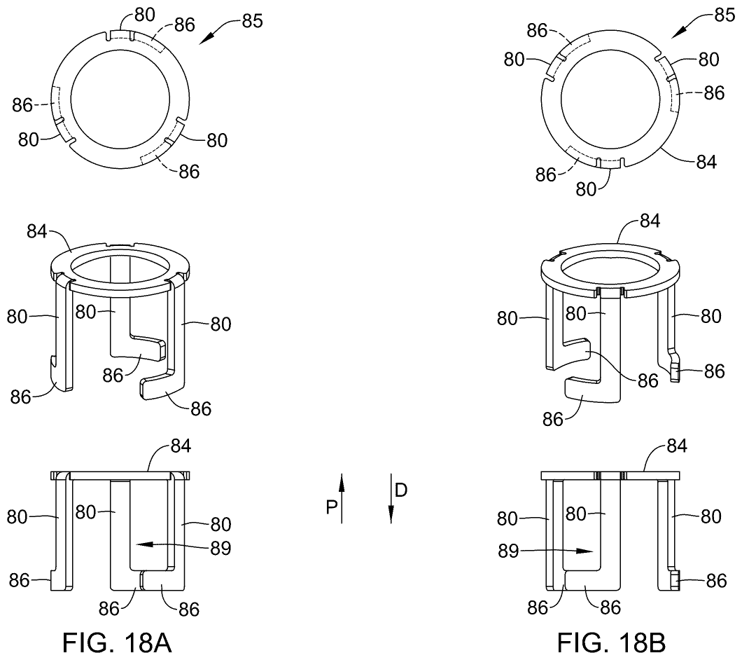

FIGS. 17, 18A, and 18B illustrate the commissure marker 85 and/or the plurality of longitudinal marker elements 80 with the annular ring 84 disposed in a distal orientation and/or spaced distally away from the stent holder 70. The plurality of longitudinal marker elements 80 extends proximally from the annular ring 84. Each longitudinal marker element of the plurality of longitudinal marker elements 80 comprises the non-longitudinal directional marker element 86. Each non-longitudinal directional marker element 86 is shown extending to the right and/or clockwise from its respective longitudinal marker element as viewed in a proximal to distal direction (e.g., from right to left in FIG. 17) along the central longitudinal axis of the elongate shaft assembly 50 (not shown in FIG. 17).

The plurality of longitudinal marker elements 80 may be disposed within the distal cap 77. Similarly, each non-longitudinal directional marker element 86 of the plurality of longitudinal marker elements 80 may be disposed within the distal cap 77. In some embodiments, the distal cap 77 may comprise a plurality of cavities disposed therein, wherein each cavity of the plurality of cavities is configured to receive one longitudinal marker element of the plurality of longitudinal marker elements 80 and/or its respective non-longitudinal directional marker element 86. In some alternative embodiments, the plurality of longitudinal marker elements 80, each non-longitudinal directional marker element 86 of the plurality of longitudinal marker elements 80, and/or the annular ring 84 may be at least partially embedded within the distal cap 77. For example, the plurality of longitudinal marker elements 80 and/or each non-longitudinal directional marker element 86 of the plurality of longitudinal marker elements 80 may be embedded within a side wall of the distal cap 77 radially inward of the radially outward facing surface 87. In some embodiments, the plurality of longitudinal marker elements 80 and/or each non-longitudinal directional marker element 86 of the plurality of longitudinal marker elements 80 may be completely embedded within the side wall of the distal cap 77. In some embodiments, the annular ring 84 may be at least partially embedded within the distal cap 77. In some embodiments, a distal face or a portion of the distal face of the annular ring 84 may be exposed from the distal cap 77. In some embodiments, the distal cap 77 may be co-molded, extruded, injection molded, etc. with and/or around the commissure marker 85.

In some embodiments, the plurality of longitudinal marker elements 80 may have a length in the longitudinal direction (e.g., proximal to distal) that is about 2 times, about 3 times, about 4 times, or about 5 times greater than a length of its corresponding non-longitudinal directional marker element 86 measured in a lateral or circumferential direction (e.g., generally perpendicular to the longitudinal direction in a side view). The difference in lengths may be useful for providing visual distinction of direction and/or orientation of the features. In some embodiments, each non-longitudinal directional marker element 86 and/or the annular ring 84 may have a thickness measured in a longitudinal direction (e.g., proximal to distal) of about 0.070 millimeters (mm) to about 0.225 mm, about 0.075 mm to about 0.200 mm, about 0.080 to about 0.175 mm, etc. Other configurations are also contemplated.

In some embodiments, the plurality of longitudinal marker elements 80 and/or each non-longitudinal directional marker element 86 of the plurality of longitudinal marker elements 80 may be disposed radially outward of the intermediate tubular member 56 (not shown in FIG. 17). In some embodiments, the plurality of longitudinal marker elements 80 and/or each non-longitudinal directional marker element 86 of the plurality of longitudinal marker elements 80 may be disposed radially outward of the body 74 of the stent holder 70. Other configurations are also contemplated. In use, the plurality of longitudinal marker elements 80, each non-longitudinal directional marker element 86 of the plurality of longitudinal marker elements 80, and the annular ring 84 would be visible under fluoroscopy, similar to FIGS. 7A-8C above. In the 3-cusp view, the commissure marker 85, the annular ring 84, and/or the plurality of longitudinal marker elements 80 would generally appear as seen in the side views of FIGS. 18A-18B, wherein FIG. 18A corresponds to the arrangement of FIG. 7A and FIG. 18B corresponds to the arrangement of FIG. 8A.

For reference, while the commissure marker 85 and the annular ring 84 are shown in the tilted views of FIGS. 18A-18B as being tilted in a forward direction (e.g., an anterior portion of the annular ring 84 is lower or more distal than a posterior portion of the annular ring 84) relative to the imaging device in the 3-cusp view, the skilled person will recognize that similar views (e.g., the annular ring 84 appearing as an oval in the 3-cusp view) may be obtained if the commissure marker 85 and the annular ring 84 are tilted in a backward direction (e.g., the posterior portion of the annular ring 84 is lower or more distal than the anterior portion of the annular ring 84) relative to the imaging device in the 3-cusp view. Any tilt in either direction could be considered to be representative of parallax.

In some embodiments, when using the commissure marker 85 with the annular ring 84, the plurality of longitudinal marker elements 80, and each non-longitudinal directional marker element 86 of the plurality of longitudinal marker elements 80 shown in FIG. 17, the replacement heart valve implant 100 (e.g., FIGS. 7C, 8C) may be properly placed within the native heart valve with respect to the native valve commissures 18 (e.g., FIGS. 7C, 8C) using only the 3-cusp view. In one example, each non-longitudinal directional marker element 86 of the plurality of longitudinal marker elements 80 may be visible under fluoroscopy in the 3-cusp view.

The direction the non-longitudinal directional marker element 86 of the middle longitudinal marker element extends may be used to identify whether the middle longitudinal marker element is disposed in the posterior position or the anterior position. For example, when the non-longitudinal directional marker element 86 of the middle longitudinal marker element extends to the right in the 3-cusp view (e.g., the side view of FIG. 18A), the middle longitudinal marker element may be disposed in the posterior position, as seen in FIG. 18A, and when the non-longitudinal directional marker element 86 of the middle longitudinal marker element extends to the left in the 3-cusp view (e.g., the side view of FIG. 18B), the middle longitudinal marker element may be disposed in the anterior position, as seen in FIG. 18B. In some alternative embodiments, these directions may be reversed, and the practitioner would be aware of the changes/differences before the procedure.

In some embodiments, the position of the middle longitudinal marker element may be determined via a configuration formed by the commissure marker 85, and/or a combination of the plurality of longitudinal marker elements 80, their non-longitudinal directional marker elements (ref. 86), and the annular ring 84. For example, in some embodiments, the annular ring 84, the plurality of longitudinal marker elements 80, and each non-longitudinal directional marker element 86 of the plurality of longitudinal marker elements 80 may form a box 89, a closed geometric feature, etc. under fluoroscopy in the 3-cusp view (e.g., the side views of FIGS. 18A-18B) such that there is no gap between two longitudinal marker elements of the plurality of longitudinal marker elements 80. When the box 89, or no gap between two longitudinal marker elements of the plurality of longitudinal marker elements 80, is disposed to the right of the middle longitudinal marker element, under fluoroscopy in the 3-cusp view (e.g., the side view of FIG. 18A), the middle longitudinal marker element may be disposed in the posterior position, and when the box 89, or no gap between two longitudinal marker elements of the plurality of longitudinal marker elements 80, is disposed to the left of the middle longitudinal marker element under fluoroscopy in the 3-cusp view (e.g., the side view of FIG. 18B), the middle longitudinal marker element may be disposed in the anterior position. In some alternative embodiments, these directions may be reversed, and the practitioner would be aware of the changes/differences before the procedure.

Accordingly, in at least some embodiments, the position of the middle longitudinal marker element may be determined without changing from the 3-cusp view. When the replacement heart valve implant 100 (e.g., FIGS. 7C, 8C) is positioned properly within the native heart valve with respect to the native valve commissures 18 (e.g., FIGS. 7C, 8C), the middle longitudinal marker element will be disposed in the posterior position.

Additionally, the annular ring 84 may provide the benefit of being able to identify parallax via visualization of forward or backward tilting of the implant delivery system 30 and/or the replacement heart valve implant 100 with respect to the imaging device in the 3-cusp view by visualizing the annular ring 84 under fluoroscopy as an oval in the 3-cusp view (e.g., the tilted views of FIGS. 18A-18B) instead of a substantially straight line. When the replacement heart valve implant 100 (e.g., FIGS. 7C, 8C) is positioned properly within the native heart valve with no parallax (e.g., no forward or backward tilt with respect to the imaging device), the annular ring 84 will appear under fluoroscopy as a substantially straight line in the 3-cusp view (e.g., the side views of FIGS. 18A-18B). Accordingly, parallax may be identified without changing to the cusp overlap view and/or using only the 3-cusp view.

In some embodiments, a method of delivering the replacement heart valve implant 100 to the native heart valve (e.g., the aortic valve 12), may comprise configuring an imaging device to produce a 3-cusp view of the native heart valve (e.g., the aortic valve 12) under fluoroscopy in a first position and a cusp overlap view of the native heart valve (e.g., the aortic valve 12) under fluoroscopy in a second position. In some alternative embodiments, two separate imaging devices may be used, with a first imaging device configured to produce the 3-cusp view under fluoroscopy and a second imaging device configuration to produce the cusp overlap view under fluoroscopy. Other configurations are also contemplated.

The method of delivering the replacement heart valve implant 100 to the native heart valve (e.g., the aortic valve 12) may comprise advancing the implant delivery system 30 to a position adjacent the native heart valve (e.g., the aortic valve 12), wherein the replacement heart valve implant 100 is constrained within the implant holding portion 60 of the implant delivery system 30. The implant delivery system 30, and/or an elongate shaft assembly 50 thereof, may comprise a plurality of longitudinal marker elements 80, as discussed herein. In at least some embodiments, each longitudinal marker element of the plurality of longitudinal marker elements 80 may comprise a non-longitudinal directional marker element 86 extending therefrom.

The method of delivering the replacement heart valve implant 100 to the native heart valve (e.g., the aortic valve 12) may comprise imaging the implant delivery system 30 adjacent the native heart valve (e.g., the aortic valve 12) under fluoroscopy with the imaging device to determine an initial orientation of the replacement heart valve implant 100 relative to the native heart valve (e.g., the aortic valve 12) in the 3-cusp view. In some embodiments, the method of delivering the replacement heart valve implant 100 to the native heart valve (e.g., the aortic valve 12) may comprise imaging the implant delivery system 30 adjacent the native heart valve (e.g., the aortic valve 12) under fluoroscopy with the imaging device to determine an initial orientation of the replacement heart valve implant 100 relative to the native heart valve (e.g., the aortic valve 12) in the 3-cusp view via relative positioning of the plurality of longitudinal marker elements 80 of the implant delivery system 30 within the native heart valve (e.g., the aortic valve 12). In at least some embodiments, it may be desirable for the plurality of posts 122 and the plurality of commissures of the replacement heart valve implant 100 to be rotationally aligned with the native valve commissures 18 of the native valve (e.g., the aortic valve 12), as shown generally in FIGS. 7A, 7C, 8A, and 8C.

In some embodiments, the method of delivering the replacement heart valve implant 100 to the native heart valve (e.g., the aortic valve 12) may comprise assessing the initial orientation under fluoroscopy in the 3-cusp view to determine if rotation of the implant delivery system 30 and the replacement heart valve implant 100 is needed to rotationally align the plurality of posts 122 and the plurality of commissures of the replacement heart valve implant 100 with the native valve commissures 18 of the native heart valve (e.g., the aortic valve 12). In some embodiments, a desired final orientation of the replacement heart valve implant 100 may include the plurality of longitudinal marker elements 80 being equally spaced apart under fluoroscopy in the 3-cusp view. If the plurality of longitudinal marker elements 80 appears to show a single longitudinal marker element of the plurality of longitudinal marker elements 80 isolated to the right of the other marker elements of the plurality of longitudinal marker elements 80 in the 3-cusp view, or a single longitudinal marker element of the plurality of longitudinal marker elements 80 isolated to the left of the other marker elements of the plurality of longitudinal marker elements 80 in the 3-cusp view, the plurality of longitudinal marker elements 80 (and thus the plurality of posts 122 and the plurality of commissures of the replacement heart valve implant 100) may be misaligned with the native valve commissures 18.

In some embodiments, the method of delivering the replacement heart valve implant 100 to the native heart valve (e.g., the aortic valve 12) may comprise, thereafter (e.g., after determining the initial orientation of the replacement heart valve implant 100), without switching from the 3-cusp view, rotating the implant delivery system 30 in situ under fluoroscopy to position the plurality of longitudinal marker elements 80 in the desired final orientation in the 3-cusp view. In the desired final orientation under fluoroscopy in the 3-cusp view, the middle longitudinal marker element of the plurality of longitudinal marker elements 80 may be disposed in the posterior position relative to other longitudinal marker elements of the plurality of longitudinal marker elements 80. In some embodiments, in the desired final orientation under fluoroscopy in the 3-cusp view, the middle longitudinal marker element of the plurality of longitudinal marker elements 80 may be disposed in the posterior position relative to other longitudinal marker elements of the plurality of longitudinal marker elements 80 as determined by the direction the non-longitudinal directional marker element (ref. 86) of and/or associated with the middle longitudinal marker element of the plurality of longitudinal marker elements 80 extends from the middle longitudinal marker element.

In some embodiments, the method of delivering the replacement heart valve implant 100 to the native heart valve (e.g., the aortic valve 12) may comprise, thereafter (e.g., after determining the initial orientation of the replacement heart valve implant 100), without switching from the 3-cusp view, rotating the implant delivery system 30 in situ under fluoroscopy to position the middle longitudinal marker of the plurality of longitudinal marker elements 80 in the posterior position relative to other longitudinal marker elements of the plurality of longitudinal marker elements 80.

In some embodiments, under fluoroscopy in the 3-cusp view, the non-longitudinal directional marker element (ref. 86) of and/or associated with the middle longitudinal marker element of the plurality of longitudinal marker elements 80 extends from the middle longitudinal marker element in a first direction when the middle longitudinal marker element of the plurality of longitudinal marker elements 80 is disposed in the posterior position relative to other longitudinal marker elements of the plurality of longitudinal marker elements 80, and the non-longitudinal directional marker element (ref. 86) of and/or associated with the middle longitudinal marker element of the plurality of longitudinal marker elements 80 extends from the middle longitudinal marker element in a second direction generally opposite the first direction when the middle longitudinal marker element of the plurality of longitudinal marker elements 80 is disposed in the anterior position relative to other longitudinal marker elements of the plurality of longitudinal marker elements 80. In some embodiments, the first direction may be left, and the second direction may be right. In some embodiments, the first direction may be right, and the second direction may be left.

In some embodiments, under fluoroscopy in the 3-cusp view, the annular ring 84 of the commissure marker 85 may be visualized as an oval if parallax is present and the annular ring 84 of the commissure marker 85 may be visualized as a straight line is parallax is not present. In at least some embodiments, the 3-cusp view may be used to determine if parallax is present without switching to the cusp overlap view.

In some embodiments, the method of delivering the replacement heart valve implant 100 to the native heart valve (e.g., the aortic valve 12) may comprise tilting the implant delivery system 30 within the native heart valve (e.g., the aortic valve 12) while visualizing the commissure marker 85 and/or the annular ring 84 under fluoroscopy in the 3-cusp view to position the replacement heart valve implant 100 in the desired final orientation, wherein in the desired final orientation the annular ring 84 of the commissure marker 85 is visualized as a straight line. In some embodiments, the method of delivering the replacement heart valve implant 100 to the native heart valve (e.g., the aortic valve 12) may comprise tilting the implant delivery system 30 within the native heart valve (e.g., the aortic valve 12) while visualizing the commissure marker 85 and/or the annular ring 84 under fluoroscopy in the 3-cusp view such that the annular ring 84 shifts from appearing as an oval to appearing as a straight line under fluoroscopy in the 3-cusp view.

In some embodiments, imaging the implant delivery system 30 adjacent the native heart valve (e.g., the aortic valve 12) under fluoroscopy with the imaging device may comprise imaging the implant delivery system 30 and the native heart valve (e.g., the aortic valve 12) in the 3-cusp view, and switching to the cusp overlap view to determine a direction of rotation of the implant delivery system 30 and the replacement heart valve implant 100 required to position the replacement heart valve implant 100 in the desired final orientation with as little rotation as possible. Rotation of the implant delivery system 30 and the replacement heart valve implant 100 in situ may cause injury to the patient's vasculature and/or the native heart valve and thus, when necessary, should be minimized to limit unnecessary risk and/or discomfort to the patient. In some embodiments, assessing the initial orientation under fluoroscopy in the 3-cusp view may include determining the direction of rotation to be counterclockwise if the plurality of longitudinal marker elements 80 appears to show a single longitudinal marker element of the plurality of longitudinal marker elements 80 isolated to the left of the other marker elements of the plurality of longitudinal marker elements 80 in the 3-cusp view. In some embodiments, assessing the initial orientation under fluoroscopy in the 3-cusp view may include determining the direction of rotation to be clockwise if the plurality of longitudinal marker elements 80 appears to show a single longitudinal marker element of the plurality of longitudinal marker elements 80 isolated to the right of the other marker elements of the plurality of longitudinal marker elements 80 in the 3-cusp view.

In some embodiments, the method of delivering the replacement heart valve implant 100 to the native heart valve (e.g., the aortic valve 12) may comprise rotating the implant delivery system 30 and the replacement heart valve implant 100 in situ to position the replacement heart valve implant 100 in the desired final orientation by aligning the plurality of longitudinal marker elements 80 with the native valve commissures 18 of the native heart valve (e.g., the aortic valve 12). In some embodiments, rotating the implant delivery system 30 and the replacement heart valve implant 100 in situ may include rotating the implant delivery system 30 and the replacement heart valve implant 100 until the plurality of longitudinal marker elements 80 appear to be equally spaced apart under fluoroscopy in the 3-cusp view, as seen in FIGS. 7A and 8A.

In some embodiments, in the desired final orientation, a single longitudinal marker element of the plurality of longitudinal marker elements 80 may be positioned farther away from the non-coronary cusp N of the native heart valve (e.g., the aortic valve 12) than other marker elements of the plurality of longitudinal marker elements 80 in the cusp overlap view, as seen in FIG. 7B. In some embodiments, the method may comprise switching back to the 3-cusp view from the cusp overlap view prior to rotating the implant delivery system 30 and the replacement heart valve implant 100 in situ. In some embodiments, the method may comprise switching back to the 3-cusp view from the cusp overlap view prior to deploying the replacement heart valve implant 100 within the native heart valve (e.g., the aortic valve 12).

In some embodiments, the method of delivering the replacement heart valve implant 100 to the native heart valve (e.g., the aortic valve 12) may comprise deploying the replacement heart valve implant 100 within the native heart valve (e.g., the aortic valve 12) with the plurality of longitudinal marker elements 80 in the desired final orientation. In some embodiments, the method of delivering the replacement heart valve implant 100 to the native heart valve (e.g., the aortic valve 12) may comprise deploying the replacement heart valve implant 100 within the native heart valve (e.g., the aortic valve 12) with the plurality of posts 122 and the plurality of commissures of the replacement heart valve implant 100 rotationally aligned with the native valve commissures 18 of the native heart valve (e.g., the aortic valve 12). Deploying the replacement heart valve implant 100 within the native heart valve (e.g., the aortic valve 12) may include radially expanding the replacement heart valve implant 100 and/or the expandable framework 110 to the radially expanded configuration within the native heart valve (e.g., the aortic valve 12) while the plurality of posts 122 and the plurality of commissures of the replacement heart valve implant 100 are rotationally aligned with the native valve commissures 18 of the native heart valve (e.g., the aortic valve 12).

In some embodiments, the method of delivering the replacement heart valve implant 100 to the native heart valve (e.g., the aortic valve 12) may comprise, prior to deploying the replacement heart valve implant 100, switching from the 3-cusp view to the cusp overlap view to verify the plurality of longitudinal marker elements 80 is disposed in the desired final orientation.

The materials that can be used for the various components of the replacement heart valve system and the various elements thereof disclosed herein may include those commonly associated with medical devices. For simplicity purposes, the following discussion refers to the system. However, this is not intended to limit the devices, components, and methods described herein, as the discussion may be applied to other elements, members, components, or devices disclosed herein, such as, but not limited to, the replacement heart valve implant, the implant delivery system, etc. and/or elements or components thereof.

In some embodiments, the system and/or components thereof may be made from a metal, metal alloy, polymer, a metal-polymer composite, ceramics, combinations thereof, and the like, or other suitable material.

Some examples of suitable polymers may include polytetrafluoroethylene (PTFE), ethylene tetrafluoroethylene (ETFE), fluorinated ethylene propylene (FEP), polyoxymethylene (POM; for example, DELRIN®), polyether block ester, polyurethane, polypropylene (PP), polyvinylchloride (PVC), polyether-ester (for example, ARNITEL®), ether or ester based copolymers (for example, butylene/poly(alkylene ether) phthalate and/or other polyester elastomers such as HYTREL®), polyamide (for example, DURETHAN® or CRISTAMID®), elastomeric polyamides, block polyamide/ethers, polyether block amide (PEBA; for example, PEBAX®), ethylene vinyl acetate copolymers (EVA), silicones, polyethylene (PE), MARLEX® high-density polyethylene, MARLEX® low-density polyethylene, linear low density polyethylene (for example, REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID®), perfluoro(propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, polyurethane silicone copolymers (for example, Elast-Eon® or ChronoSil®), biocompatible polymers, other suitable materials, or mixtures, combinations, copolymers thereof, polymer/metal composites, and the like. In some embodiments, the system and/or components thereof can be blended with a liquid crystal polymer (LCP). For example, the mixture can contain up to about 6 percent LCP.

Some examples of suitable metals and metal alloys include stainless steel, such as 304 and/or 316 stainless steel and/or variations thereof; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; platinum; palladium; gold; combinations thereof; or any other suitable material.

In at least some embodiments, portions or all of the system and/or components thereof may also be doped with, made of, or otherwise include a radiopaque material. Radiopaque materials are understood to be materials capable of producing a relatively dark image on a fluoroscopy screen or another imaging technique (e.g., ultrasound, etc.) during a medical procedure. This relatively dark image aids the user of the system in determining its location. Some examples of radiopaque materials can include, but are not limited to, gold, platinum, palladium, tantalum, tungsten and alloys thereof, polymer material loaded with a radiopaque filler, and the like. Additionally, other radiopaque marker bands and/or coils may also be incorporated into the design of the system to achieve the same result.

In some embodiments, a degree of Magnetic Resonance Imaging (MRI) compatibility is imparted into the system and/or other elements disclosed herein. For example, the system and/or components or portions thereof may be made of a material that does not substantially distort the image and create substantial artifacts (e.g., gaps in the image). Certain ferromagnetic materials, for example, may not be suitable because they may create artifacts in an MRI image. The system or portions thereof may also be made from a material that the MRI machine can image. Some materials that exhibit these characteristics include, for example, tungsten, cobalt-chromium-molybdenum alloys (e.g., UNS: R30003 such as ELGILOY®, PHYNOX®, and the like), nickel-cobalt-chromium-molybdenum alloys (e.g., UNS: R30035 such as MP35-N® and the like), nitinol, and the like, and others.

In some embodiments, the system and/or other elements disclosed herein may include a fabric material disposed over or within the structure. The fabric material may be composed of a biocompatible material, such a polymeric material or biomaterial, adapted to promote tissue ingrowth. In some embodiments, the fabric material may include a bioabsorbable material. Some examples of suitable fabric materials include, but are not limited to, polyethylene glycol (PEG), nylon, polytetrafluoroethylene (PTFE, ePTFE), a polyolefinic material such as a polyethylene, a polypropylene, polyester, polyurethane, and/or blends or combinations thereof.

In some embodiments, the system and/or other elements disclosed herein may include and/or be formed from a textile material. Some examples of suitable textile materials may include synthetic yarns that may be flat, shaped, twisted, textured, pre-shrunk or un-shrunk. Synthetic biocompatible yarns suitable for use in the present disclosure include, but are not limited to, polyesters, including polyethylene terephthalate (PET) polyesters, polypropylenes, polyethylenes, polyurethanes, polyolefins, polyvinyls, polymethylacetates, polyamides, naphthalene dicarboxylene derivatives, natural silk, and polytetrafluoroethylenes. Moreover, at least one of the synthetic yarns may be a metallic yarn or a glass or ceramic yarn or fiber. Useful metallic yarns include those yarns made from or containing stainless steel, platinum, gold, titanium, tantalum, or a Ni—Co—Cr-based alloy. The yarns may further include carbon, glass, or ceramic fibers. Desirably, the yarns are made from thermoplastic materials including, but not limited to, polyesters, polypropylenes, polyethylenes, polyurethanes, polynaphthalenes, polytetrafluoroethylenes, and the like. The yarns may be of the multifilament, monofilament, or spun types. The type and denier of the yarn chosen may be selected in a manner which forms a biocompatible and implantable prosthesis and, more particularly, a vascular structure having desirable properties.

In some embodiments, the system and/or other elements disclosed herein may include and/or be treated with a suitable therapeutic agent. Some examples of suitable therapeutic agents may include anti-thrombogenic agents (such as heparin, heparin derivatives, urokinase, and PPack (dextrophenylalanine proline arginine chloromethyl ketone)); anti-proliferative agents (such as enoxaparin, angiopeptin, monoclonal antibodies capable of blocking smooth muscle cell proliferation, hirudin, and acetylsalicylic acid); anti-inflammatory agents (such as dexamethasone, prednisolone, corticosterone, budesonide, estrogen, sulfasalazine, and mesalamine); antineoplastic/antiproliferative/anti-mitotic agents (such as paclitaxel, 5-fluorouracil, cisplatin, vinblastine, vincristine, epothilones, endostatin, angiostatin and thymidine kinase inhibitors); anesthetic agents (such as lidocaine, bupivacaine, and ropivacaine); anti-coagulants (such as D-Phe-Pro-Arg chloromethyl ketone, an RGD peptide-containing compound, heparin, anti-thrombin compounds, platelet receptor antagonists, anti-thrombin antibodies, anti-platelet receptor antibodies, aspirin, prostaglandin inhibitors, platelet inhibitors, and tick antiplatelet peptides); vascular cell growth promoters (such as growth factor inhibitors, growth factor receptor antagonists, transcriptional activators, and translational promoters); vascular cell growth inhibitors (such as growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin); immunosuppressants (such as the “olimus” family of drugs, rapamycin analogues, macrolide antibiotics, biolimus, everolimus, zotarolimus, temsirolimus, picrolimus, novolimus, myolimus, tacrolimus, sirolimus, pimecrolimus, etc.); cholesterol-lowering agents; vasodilating agents; and agents which interfere with endogenous vasoactive mechanisms.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The scope of the disclosure is, of course, defined in the language in which the appended claims are expressed.

What is claimed is:

1. An implant delivery system for delivering a replacement heart valve implant to a native heart valve, comprising:
an elongate shaft assembly including an implant holding portion comprising a proximal sheath and a distal sheath;
wherein the implant holding portion is configured to constrain a replacement heart valve implant in a radially collapsed configuration;
wherein the elongate shaft assembly comprises:
a stent holder configured to engage an expandable framework of the replacement heart valve implant in the radially collapsed configuration; and
a plurality of longitudinal marker elements configured to be visible under fluoroscopy with an imaging device, the plurality of longitudinal marker elements being spaced apart distally from the stent holder;
wherein the plurality of longitudinal marker elements is configured to rotationally align commissure posts of the replacement heart valve implant with native valve commissures of the native heart valve under fluoroscopy with the imaging device when the replacement heart valve implant is shifted to a radially expanded configuration within the native heart valve.

2. The implant delivery system of claim 1, wherein each longitudinal marker element of the plurality of longitudinal marker elements comprises a non-longitudinal directional marker element extending therefrom.

3. The implant delivery system of claim 2, wherein each longitudinal marker element of the plurality of longitudinal marker elements is oriented parallel to a central longitudinal axis of the elongate shaft assembly and each non-longitudinal directional marker element extends laterally from its respective longitudinal marker element.

4. The implant delivery system of claim 2, wherein each longitudinal marker element of the plurality of longitudinal marker elements is oriented parallel to a central longitudinal axis of the elongate shaft assembly and each non-longitudinal directional marker element extends circumferentially from its respective longitudinal marker element.

5. The implant delivery system of claim 2, wherein each non-longitudinal directional marker element extends from a free end of its respective longitudinal marker element.

6. The implant delivery system of claim 2, wherein each non-longitudinal directional marker element extends from a medial portion of its respective longitudinal marker element.

7. The implant delivery system of claim 1, wherein each longitudinal marker element of the plurality of longitudinal marker elements is formed from a radiopaque material and the stent holder is formed from a different material than the plurality of longitudinal marker elements.

8. The implant delivery system of claim 1, wherein each longitudinal marker element of the plurality of longitudinal marker elements is fixedly attached to an annular ring that is disposed about a central longitudinal axis of the elongate shaft assembly.

9. The implant delivery system of claim 8, wherein the annular ring is spaced apart distally from the stent holder.

10. An implant delivery system for delivering a replacement heart valve implant to a native heart valve, comprising:
an elongate shaft assembly including an implant holding portion comprising a proximal sheath and a distal sheath;
wherein the implant holding portion is configured to constrain a replacement heart valve implant in a radially collapsed configuration;
wherein the elongate shaft assembly comprises:
a stent holder configured to engage a distal portion of an expandable framework of the replacement heart valve implant when the replacement heart valve implant is constrained within the implant holding portion in the radially collapsed configuration;
a plurality of longitudinal marker elements disposed distal of the stent holder and configured to be visible under fluoroscopy; and
a distal cap disposed at least partially distal of the stent holder and extending distal of the plurality of longitudinal marker elements;
wherein the plurality of longitudinal marker elements is disposed radially inward of an outward facing surface of the distal cap;
wherein the plurality of longitudinal marker elements is configured to rotationally align commissure posts of the replacement heart valve implant with native valve commissures of the native heart valve under fluoroscopy with the imaging device when the replacement heart valve implant is shifted to a radially expanded configuration within the native heart valve.

11. The implant delivery system of claim 10, wherein each longitudinal marker element of the plurality of longitudinal marker elements is fixedly attached to an annular ring disposed about a central longitudinal axis of the elongate shaft assembly.

12. The implant delivery system of claim 11, wherein each longitudinal marker element of the plurality of longitudinal marker elements extends distally from the annular ring.

13. The implant delivery system of claim 11, wherein each longitudinal marker element of the plurality of longitudinal marker elements extends proximally from the annular ring.

14. The implant delivery system of claim 11, wherein the distal cap is substantially transparent under fluoroscopy.

15. A method of delivering a replacement heart valve implant to a native heart valve, comprising:
configuring an imaging device to produce a 3-cusp view of the native heart valve under fluoroscopy in a first position and a cusp overlap view of the native heart valve under fluoroscopy in a second position;
advancing an implant delivery system to a position adjacent the native heart valve, wherein the replacement heart valve implant is constrained within an implant holding portion of the implant delivery system, and the implant delivery system comprises a plurality of longitudinal marker elements;
wherein each longitudinal marker element of the plurality of longitudinal marker elements comprises a non-longitudinal directional marker element extending therefrom;
imaging the implant delivery system adjacent the native heart valve under fluoroscopy with the imaging device to determine an initial orientation of the replacement heart valve implant relative to the native heart valve in the 3-cusp view;
thereafter, without switching from the 3-cusp view, rotating the implant delivery system in situ under fluoroscopy to position the plurality of longitudinal marker elements in a desired final orientation in the 3-cusp view, wherein in the desired final orientation under fluoroscopy in the 3-cusp view a middle longitudinal marker element of the plurality of longitudinal marker elements is disposed in a posterior position relative to other longitudinal marker elements of the plurality of longitudinal marker elements; and
deploying the replacement heart valve implant within the native heart valve with the plurality of longitudinal marker elements in the desired final orientation.

16. The method of claim 15, further comprising:
prior to deploying the replacement heart valve implant, switching to the cusp overlap view to verify the plurality of longitudinal marker elements is in the desired final orientation.

17. The method of claim 16, wherein under fluoroscopy in the desired final orientation, a single longitudinal marker element of the plurality of longitudinal marker elements is positioned farther away from a non-coronary cusp of the native heart valve than other longitudinal marker elements of the plurality of longitudinal marker elements in the cusp overlap view.

18. The method of claim 15, wherein under fluoroscopy in the 3-cusp view, the non-longitudinal direction marker element associated with the middle longitudinal marker element of the plurality of longitudinal marker elements extends from the middle longitudinal marker element in a first direction when the middle longitudinal marker element is disposed in the posterior position relative to other longitudinal marker elements of the plurality of longitudinal marker elements, and the non-longitudinal direction marker element associated with the middle longitudinal marker element of the plurality of longitudinal marker elements extends from the middle longitudinal marker element extends in a second direction generally opposite the first direction when the middle longitudinal marker element is disposed in an anterior position relative to other longitudinal marker elements of the plurality of longitudinal marker elements.

19. The method of claim 15, wherein each longitudinal marker element of the plurality of longitudinal marker elements is fixedly attached to an annular ring formed from a radiopaque material.

20. The method of claim 19, wherein under fluoroscopy in the 3-cusp view, the annular ring is visualized as an oval if parallax is present and the annular ring is visualized as a straight line is parallax is not present.
